(19) United States
(12) Patent Application Publication
(10) Pub. No.: US 2004/0266742 A1
(43) Pub. Date: Dec. 30, 2004

(54) Combination therapy of an sodm and a corticosteroid for prevention and/or treatment of inflammatory disease

(76) Inventor: Daniela Salvemini, Chesterfield, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 10/481,396

(22) PCT Filed: Jun. 26, 2002
(86) PCT No.: PCT/US02/20476

Related U.S. Application Data
(60) Provisional application No. 60/301,080, filed on Jun. 26, 2001.

(51) Int. Cl. A61K 31/573; A61K 31/555

(52) U.S. Cl. 514/171; 514/184

(57) ABSTRACT

The present invention relates to pharmaceutical compositions and methods using such compositions for the treatment of inflammatory disease. Such compositions contain a catalyst for the dismutation of superoxide, including superoxide dismutase enzyme (SOD) and low molecular weight organic ligand derived metal complexes characterized in having the following structure: (Z)ₙ that function as mimics of the enzyme (SOD mimetics or SODm) in combination with corticosteroids.

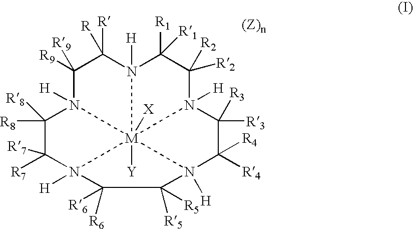

This application claims the benefit of U.S. Provisional Application No. 60/301,080, filed Jun. 26, 2001, herein incorporated by reference in its entirety.

### FIELD OF THE INVENTION

The present invention relates to the use of a combination of steroids with compounds which are effective as catalysts for dismutating superoxide and, more particularly, the manganese or iron complexes of substituted, unsaturated heterocyclic pentaazacyclopentadecane ligands which catalytically dismutate superoxide.

### BACKGROUND OF THE INVENTION

Inflammatory disease is any disease marked by inflammation, which is a localized protective response elicited by injury or destruction of tissues and serves to destroy, dilute, or separate both the injurious agent and the injured tissue. Inflammation is characterized in the acute form by the classical signs of pain, heat, redness, swelling and loss of function. Inflammation occurs when, upon injury, recruited polymorphonuclear leukocytes release Reactive Oxygen Species (ROS) in oxidative bursts resulting in a complex cascade of events. Histologically, it involves a complex series of events, including dilation of arterioles, capillaries, and venules, with increased permeability and blood flow; exudation of fluids, including plasma proteins; and leukocytic migration into the inflammatory focus. One of the most prominent of inflammatory diseases is arthritis, which refers to inflammation of the joints. Other inflammatory diseases include inflammatory bowel disease, asthma, psoriasis, lupus and other autoimmune diseases. The inflammation of the inflammatory diseases may be caused by a multitude of inciting events, including radiant, mechanical, chemical, infectious, and immunological stimuli;

One of the most prominent inflammatory diseases is arthritis. Arthritis is a term that refers to a group of more than 100 diseases that cause joint swelling, tissue damage, stiffness, pain (both acute and chronic), and fever. Arthritis can also affect other parts of the body other than joints including but not limited to: synovium, joint space, collagen, bone, tendon, muscle and cartilage, as well as some internal organs. The two most common forms of arthritis are osteoarthritis (“OA”) and rheumatoid arthritis (“RA”). RA is the most severe of these two forms in terms of pain, while OA is the most common form. Rheumatoid arthritis is a systematic, inflammatory, autoimmune disease that commonly affects the joints, particularly those of the hands and feet. Autoimmune diseases are caused by an abnormal immune response involving either cells or antibodies directed against normal tissues. A number of strategies have been developed to suppress autoimmune diseases, most notably drugs which nonspecifically suppress the immune response. The onset of rheumatoid arthritis can occur slowly, ranging from a few weeks to a few months, or the condition can surface rapidly in an acute manner.

At the cellular level, inflammatory diseases are characterized by an accumulation of cytokines such as TNF-α, IL-1β, IL-6, IL-9, IL-11, IL-15, IL-5 and several belonging to the interferon family, as well as inflammatory cells (e.g., eosinophils, neutrophils, and macrophages). Focussing on arthritis specifically, these cytokines accumulate in synovial fluid during arthritic flare-up. Many of these cytokines are released from inflammatory cells which in turn cause cell and tissue damage. Additionally, another significant characteristic of the inflammatory response associated with arthritis and other diseases like lupus is a process called autoimmunity. Autoimmunity occurs when T-cells mistake the body's own collagen cells as foreign antigens and set off a series of events to clear the erroneously perceived threat. This results in an attack of the body's own cells by its immune system. Autoimmunity is particularly associated with rheumatoid arthritis and lupus. The immune response associated with arthritic flare-up is also characterized by oxidative and nitrosative stress and polyADP-ribose synthetase (PARS) activity.

Aspirin is widely used to treat pain and to reduce inflammation in many inflammatory diseases. In addition to aspirin, non-steroidal anti-inflammatory drugs, corticosteroids, gold salts, anti-malarials and systemic immunosuppressants are widely used in moderate to advanced cases of arthritis and other inflammatory diseases. Corticosteroids are a very effective drug for the treatment of arthritis, other inflammatory diseases and the pain associated with these disease and are the most potent anti-inflammatory agents previously known.

Corticosteroids have 21 carbon atoms and are classified as glucocorticoids and mineralocorticoids. The effects of corticosteroids are numerous and widespread. Some of these effects include: alterations in carbohydrate, protein, and lipid metabolism; maintenance of fluid and electrolyte balance; and preservation of normal function of the cardiovascular system, the immune system, the kidney, skeletal muscle, the endocrine system, and the nervous system. The mechanisms of corticosteroids are still not fully understood, but corticosteroids endow the organism with the capacity to resist stressful circumstances such as noxious stimuli and environmental changes. One of the major pharmaceutical uses for corticosteroids are as anti-inflammatory and immunosuppressive agents. The pharmacological actions of corticosteroids in different tissues and many of their physiological effects seem to be mediated by the same receptor. The corticosteroid receptor is deactivated by superoxide and by peroxynitrite. See Macarthur et al., Inactivation of Catecholamines by Superoxide Gives New Insights on the Pathogenesis of Septic Shock, PNAS, Vol. 97, No. 17, 9753-9758 (Aug. 15, 2000). Thus, the various glucocorticoid derivatives used as pharmacological agents have side effects on physiological processes that parallel their therapeutic effectiveness.

The actions of corticosteroids are related in complex ways to those of other hormones. Corticosteroids interact with specific receptor proteins in target tissues to regulate the expression of corticosteroid-responsive genes, thereby changing the levels and variety of proteins synthesized by the various target tissues. Corticosteroids profoundly alter the immune responses of lymphocytes having an important effect on the anti-inflammatory and immunosuppressive actions of the body. The immunosuppressive and anti-inflammatory actions of glucocorticoids are inextricably linked, perhaps because they both largely result from inhibition of specific functions of leukocytes.

For many years corticosteroids have been used for treating inflammatory conditions. Generally, prednisone, an alcohol, is used orally, and the corresponding ketone prednisolone (or methyl-prednisolone) is used for parenteral injections. These compounds are five times more effective than naturally occurring cortisone and thus minimize toxicity problems. Later-developed fluorinated derivatives of corticosteroids (e.g., triamcinolone, dexamethasone, paramethasone, and betamethasone) came into use, which are three to five times more effective than non-fluorinated compounds, but are also more toxic. Corticosteroids are the most widely used anti-inflammatory drugs for both acute and chronic inflammation. They are used orally, parenterally, and frequently, intra- and peri-articularly, i.e., injections in and around joints and joint cavities. However, the side effects associated with corticosteroid use can be severe.

ROS include the superoxide anion (O₂⁻), hydroxyl radical (OH⁻), and nitric oxide (NO⁻) as well as other species. ROS metabolites derived from the superoxide anion are postulated to contribute to the tissue pathology in a number of inflammatory diseases, such as reperfusion injury (particularly for the intestine, liver, heart and brain), inflammatory bowel disease, rheumatoid arthritis, osteoarthritis, atherosclerosis, hypertension, cancer, skin disorders (e.g., psoriasis, dermatitis), organ transplant rejections, chemotherapy and radiation-induced side effects, pulmonary disorders (e.g., chronic obstructive pulmonary disease (COPD), asthma), influenza, stroke, burns, AIDS, malaria, Parkinson's disease and trauma. See, for example, Simic, M. G., et al, “Oxygen Radicals in Biology and Medicine”, Basic Life Sciences, Vol. 49, Plenum Press, New York and London, 1988; Weiss J. Cell. Biochem., 1991 Suppl. 15C, 216 Abstract C110 (1991); Petkau, A., Cancer Treat. Rev. 13, 17 (1986); McCord, J. Free Radicals Biol. Med., 2, 307 (1986); and Bannister, J. V. et al, Crit. Rev. Biochem., 22, 111 (1987). ROS contribute significantly to tissue injury in Rheumatoid arthritis and other inflammatory diseases. See Bauerova et al., “Role of Reactive Oxygen and Nitrogen Species in Etiopathogenesis of Rheumatiod Arthritis” Gen Physiol Biophys 1999 October; 18 Spec No.: 15-20.

ROS are produced in vivo through normal cellular respiration and natural biological signaling and defense mechanisms. Although cellular respiration is important to maintaining life, these highly reactive byproduct molecules have been implicated in a wide range of diseases and conditions. For example, during inflammation, recruited polymorphonuclear leukocytes release ROS during the oxidative burst of phagocytosis. However, during chronic and/or systemic inflammation, the body's ability to control the levels of ROS, specifically superoxide anion radicals, becomes overwhelmed. Llesuy et al., Free Radical Biology and Medicine, 16(4), 445451 (1994); Taylor et al., Journal of Critical Care, 10(3), 122-136 (1995). The rampant oxidative stress that occurs during the stage of sepsis quickly reduces the levels and/or activities of the body's natural antioxidants (e.g., ascorbate, superoxide dismutase, catalase, glutathione peroxidase, vitamin E) and lipid peroxides begin to accumulate. Additionally, endogenous catecholamines and cortisol may be inactivated leading to a drop in blood pressure and an increase in vascular permeability. See Macarthur et al., Inactivation of Catecholamines by Superoxide Gives New Insights on the Pathogenesis of Septic Shock, PNAS, Vol. 97, No. 17, 9753-9758 (Aug. 15, 2000).

Physiologically, glucocorticoids are produced by the adrenal cortex and regulate carbohydrate metabolism, embryogenic organ development, and immunosuppression. See de Waal, R. M. W. Molecular Biology Reports, 1994, 19, 81-88; Schimmer et al., The Pharmacological Basis of Therapeutics, 9th ed., Hardman et al., McGraw-Hill: New York, 1996; Chap. Pharmacologically, they are the most widely used immunosuppressive drugs and are the most potent anti-inflammatory agents previously known. The pharmacological effects of glucocorticoids appear to be mediated by the same receptor resulting in side effects that parallel their therapeutic effectiveness. However, glucocorticoid side-effect profiles occur at doses much lower than those required for an anti-inflammatory effect. In addition, some glucocorticoids possess modest mineralocorticoid activity including maintenance of fluid and electrolyte balance. As a result, corticosteroids are typically compared by ranking their anti-inflammatory (gluco-) and sodium retaining (mineralo-) potencies. The table below demonstrates the relative potencies and equivalent doses of representative corticosteroids.

TABLE 1
Relative Potencies and Equivalent Doses of Representative Corticosteroids

| | Anti- | Na⁺- | | |
|---|---|---|---|---|
| | inflammatory | retaining | Duration | Equivalent |
| Compound | Potency | Potency | (T_{½}, Hours) | Dose (mg) |
| Cortisol | 1 | 1 | 8-12 | 20 |
| Cortisone | 0.8 | 0.8 | 8-12 | 25 |
| Fludrocortisone | 10 | 12.5 | 8-12 | N/A |
| Prednisone | 4 | 0.8 | 12-36 | 5 |
| Prednisolone | 4 | 0.8 | 12-36 | 5 |
| 6a-Methyl- | 5 | 0.5 | 12-36 | 4 |
| prednisolone | | | | |
| Triamcinolone | 5 | 0 | 12-36 | 4 |
| Betamethasone | 25 | 0 | 36-72 | 0.75 |
| Dexamethasone | 25 | 0 | 36-72 | 0.75 |

Because glucocorticoid effects are mediated by the same glucocorticoid receptor, SAR chemistry has had limited success in separating anti-inflammatory efficacy from fluid and electrolyte abnormalities, hypertension, hyperglycemia, increased susceptibility to infection, osteonecrosis, osteoporosis, myopathy, behavioral disturbances, cataracts, growth arrest, fat redistribution, striae, ecchymoses, acne, and hirsutism. Classical SAR theory maintains that there are five critical functionalities for glucocorticoid receptor agonism: the 3-oxo, the Δ⁴-ene, the 11-hydroxy, the 19-methyl, and the 20-carbonyl. It is implied that the loss of any one of these structures results in significant to complete loss of anti-inflammatory activity. However, there are several reports of active anti-inflammatory steroids lacking these minimal functionalities that bind to the glucocorticoid receptor and possess like or significantly decreased side effects. These few examples may provide added enthusiasm in investigating new glucocorticoids and pro-drugs and/or may question the explicitness of the receptor-mediated mechanism.

Superoxide anions are normally removed in biological systems by the formation of hydrogen peroxide and oxygen in the following reaction (hereinafter referred to as dismutation):

O₂⁻+O₂⁻+2H⁺→O₂+H₂O₂

This reaction is catalyzed in vivo by the ubiquitous superoxide dismutase (SOD) enzyme. This reaction represents the mechanism by which naturally occurring SOD or a SOD mimetic catalyzes superoxide for the purposes of this invention.

It is also known that O₂⁻ is involved in the breakdown of proteins, lipids, DNA, uric acid, polysaccharides, which have been shown to be increased in RA patients. These proteins, lipids, DNA, uric acid, and polysaccharides are protected from breakdown by SOD. Also, reactive oxygen species are directly involved in tissue injuries and indirectly facilitate tissue destruction by inactivating α-1-protease inhibitors that form a complex with elastase, a serine proteinase. Bauerova et al., Role of Reactive Oxygen and Nitrogen Species in Etiopathogenesis of Rheumatoid Arthritis, Gen. Physiol. Biophys. 18, Focus Issue, 15-20 (1999). Studies have shown that chondrocyte-derived ROS damage cartilage matrix and mediate matrix degradation as part of the pathogenesis of both cartilage aging and OA. Tiku et al., Evidence Linking Chondrocyte Lipid Peroxidation to Cartilage Matrix Protein Degradation, J. Biol. Chem., Vo. 275, No. 26, 20069-20076 (Jun. 30, 2000); Mattey et al., Influence of Polymorphism in the Manganese Superoxide Dismutase Locus on Disease Outcome in Rheumatoid Arthritis, Arthritis & Rheumatism, Vol. 43, No. 4, 859-864 (April 2000).

ROS have also been implicated in the damage of hyaluronic acid (HA), which is depolymerized causing synovial fluid to lose its lubricating properties causing friction in the joint. Kataoka et al., Hydroxyl radical scavenging activity of nonsteroidal antiinflammatory drugs, Free Radical Res. 27, 419427 (1997). Hyaluronan attacked by ROS yields several intermediates and end-products found in increased concentrations in the synovial fluid and serum of rheumatic patients. Orvisky et al., High-molecular-weight hyaluronan a valuable tool in testing the antioxidative activity of amphiphilic drugs stobadine and vinpocetine, J. Pharm. Biomed. Anal. 16, 419-424 (1997); Mertens, et al., Study of eosinophil-endothelial adhesion, production of oxygen radicals and release of eosinophil cationic protein by peripheral blood eosinophils of patients with rheumatoid arthritis, Clinical and Experimental Allergy, Vol. 23, 868-873 (1993). This suggests a central role for activated oxygen species derived from superoxide in the pathogenesis RA. See, for example, Bauerova et al., Role of Reactive Oxygen and Nitrogen Species in Etiopathogenesis of Rheumatoid Arthritis, Gen. Physiol. Biophys., 18, 15-20 (1999).

Recently, a class of non-peptidic, low-molecular weight possessing a catalytic activity and high selectivity comparable to native SOD have been reported and the use of these compounds has been suggested for assessing a better therapeutic approach in diseases mediated by superoxide overproduction (Salvemini et al., Science 8, 304-306 (1999)). Several non-peptidic catalysts which mimic this superoxide dismutating activity have been discovered. A particularly effective family of non-peptidic catalysts for the dismutation of superoxide consists of the manganese(II), manganese(III), iron(II) or iron(III) complexes of nitrogen-containing fifteen-membered macrocyclic ligands which catalyze the conversion of superoxide into oxygen and hydrogen peroxide, as described in U.S. Pat. Nos. 5,874,421 and 5,637,578, all of which are incorporated herein by reference. See also, Weiss, R. H., et al., “Manganese(II)-Based Superoxide Dismutase Mimetics: Rational Drug Design of Artificial Enzymes”, Drugs of the Future 21: 383-389 (1996); and Riley, D. P., et al., “Rational Design of Synthetic Enzymes and Their Potential Utility as Human Pharmaceuticals” (1997) in CatTech, I, 41. These mimics of superoxide dismutase have been shown to have a variety of therapeutic effects, including anti-inflammatory activity. See Weiss, R. H., et al., “Therapeutic Aspects of Manganese (II)-Based Superoxide Dismutase Mimics” In “Inorganic Chemistry in Medicine”, (Farrell, N., Ed.), Royal Society of Chemistry, in Press; Weiss, R. H., et al., “Manganese-Based Superoxide Dismutase Mimics: Design, Discovery and Pharmacologic Efficacies” (1995), In “The Oxygen Paradox” (Davies, K. J. A., and Ursini, F., Eds.) pp. 641-651, CLEUP University Press, Padova, Italy; Weiss, R. H., et al., J. Biol. Chem., 271: 26149 (1996); and Hardy, M. M., et al., J. Biol. Chem. 269: 18535-18540 (1994). Other non-peptidic catalysts which have been shown to have superoxide dismutating activity are complexes of porphyrins with iron and manganese cations.

Clinical trials and animal studies with natural, recombinant and modified SOD have been completed or are ongoing to demonstrate the therapeutic efficacy of reducing superoxide levels in the disease states noted above. However, numerous problems arise with the use of these enzymes as potential therapeutic agents, including lack of oral activity, short half-lives in vivo, immunogenicity with nonhuman derived enzymes, and poor tissue distribution.

Thus, the need presently exists for effective compositions and methods for preventing and treating inflammatory disorders including RA associated with the overproduction of ROS. In addition, there is a need for increasing the effectiveness of glucocorticoids, which may be deactivated by free radicals, for the treatment of inflammatory disease.

### SUMMARY OF THE INVENTION

Other features of the present invention will be in part apparent to those skilled in the art and in part pointed out in the detailed description provided below.

The present invention provides a method for treating inflammatory disease in a subject comprising co-administering a therapeutically effective amount to the subject of a catalyst for the dismutation of superoxide in conjunction with at least one corticosteroid.

The present invention further provides a method for treatment of arthritis, the method comprising co-administering to a subject a therapeutically effective amount of a composition comprising a non-proteinaceous catalyst for the dismutation of superoxide anions and at least one corticosteroid.

Additionally, the present invention provides a pharmaceutical composition for the treatment of inflammatory disease comprising a non-proteinaceous catalyst for the dismutation of superoxide anions, a corticosteroid and a pharmaceutically acceptable carrier.

The present invention also provides a combination comprising a non-proteinaceous catalyst and a corticosteroid, wherein said non-proteinaceous catalyst and corticosteroid together comprise a therapeutically effective amount of said non-proteinaceous catalyst and corticosteroid.

In addition, a kit comprising at least one non-proteinaceous catalyst and at least one corticosteroid is provided.

### BRIEF DESCRIPTION OF THE DRAWINGS

These and other features, aspects, and advantages of the present invention will become better understood with regard to the following description, appended claims and accompanying figures where:

FIG. 1. Effect of combination therapy (dexamethasone (DEX) 0.01 mg/kg+M40403 2 mg/kg) on the onset of collagen-induced arthritis. The percentage of arthritic rats (rats showing clinical scores of arthritis are shown in panel (A). Median arthritic score during collagen-induced arthritis is shown in panel (B). Values are means i standard error of the mean (s.e.m.) of 10 animals for each group. *p<0.01 versus Control. °p<0.01 versus CIA.

FIG. 2. Effect of combination therapy (DEX 0.01 mg/kg+M40403 2 mg/kg) on paw swelling. Values are means i s.e.m. of 10 animals for each group. *p<0.01 versus Control. °p<0.01 versus CIA.

FIG. 3. Plasma levels of TNF-α (A) and IL-1β (B). Cytokine levels were significantly reduced in the plasma from rats which received DEX (0.1 mg/kg) or combination therapy (DEX 0.01 mg/kg+M40403 2 mg/kg). Values are means±s.e.m. of 10 animals for each group. *p<0.01 versus sham. °p<0.01 versus CIA.

FIG. 4. Effect of combination therapy (DEX 0.01 mg/kg+M40403 2 mg/kg) malondialdehyde (MDA) levels in plasma: MDA levels in the plasma of CII-immunized rats killed at 35 days. MDA levels were significantly increased in the plasma of the CII-immunized rats in comparison to sham rats (*p<0.01). DEX (0.1 mg/kg) or combination therapy (DEX 0.01 mg/kg+M40403 2 mg/kg) reduced the CIA increase in MDA levels. Values are means±s.e.m. of 10 rats for each group. *p<0.01 versus shamp. °p<0.01 versus CIA.

FIG. 5. Nitrotyrosine immunostaining in the paw of a control rat (A) and the paw of a rat at 35 days of collagen-induced arthritis (B). A marked increase in Nitrotyrosine staining is evident in the paws in arthritis. There was a marked reduction in the immunostaining in the paw of rats which were treated with DEX (0.1 mg/kg) (C) or with combination therapy (DEX 0.01 mg/kg+M40403 2 mg/kg) (D). Original magnificantion: X125. Figure is representative of at least 3 experiments performed on different experimental days.

FIG. 6. Effect of combination therapy (DEX 0.01 mg/kg+M40403 2 mg/kg) on PARS activity: Staining was absent in control tissue (A). 35 days following collagen-induced arthritis, PARS immunoreactivity was present in the paw from CII-immunized rats (B). There was a marked reduction in the immunostaining in the paw of rats which were treated with DEX (0.1 mg/kg) (C), or combination therapy (DEX 0.01 mg/kg+M40403 2 mg/kg) (D) no positive staining was found. Original magnification: X125. Figure is representative of at least 3 experiments performed on different experimental days.

FIG. 7. Plasma levels of nitrite/nitrate (NOₓ). NOₓ levels were significantly reduced in the plasma from rats which received DEX (0.1 mg/kg) or combination therapy (DEX 0.01 mg/kg+M40403 2 mg/kg). Values are means±s.e.m. of 10 animals for each group. *p<0.01 versus sham. °p<0.01 versus CIA.

FIG. 8. Inducible nitric oxide synthase (iNOS) immunostaining in the paw of a control rat (A) and the paw of a rat at 35 days of collagen-induced arthritis (B). A marked increase in iNOS staining is evident in the paws afflicted with arthritis. There was a marked reduction in the immunostaining in the paw of rats which were treated with DEX (0.1 mg/kg) (C) or a combination therapy (DEX 0.01 mg/kg+M40403 2 mg/kg) (D). Original magnification: X125. Figure is representative of at least 3 experiments performed on different experimental days.

FIG. 9. Effect of combination therapy (DEX 0.01 mg/kg+M40403 2 mg/kg) on COX-2 expression: Staining was absent in control tissue (A). 35 days following collagen-induced arthritis, COX-2 immunoreactivity was present in the paw from CII-immunized rats (B). In the paw of rats which received DEX (0.1 mg/kg) (C), or combination therapy (DEX 0.01 mg/kg+M40403 2 mg/kg) (D) no positive staining was found. Original magnification: X125. Figure is representative of at least 3 experiments performed on different experimental days.

FIG. 10. Effect of combination therapy (DEX 0.01 mg/kg+M40403 2 mg/kg) on body weight gain. Beginning on day 25, the collagen-challenged rats or rats treated with low doses of DEX (0.01 mg/kg) or M40403 (2 mg/kg) alone gained significantly less weight than the normal rats, and this trend continued through day 35. On the other hand, DEX at the high dose tested (0.1 mg/kg) or combination of low doses DEX and M40403 (0.01 mg/kg+2 mg/kg respectively) gained weight in a manner similar to sham animals. Values are means±s.e.m. of 10 animals for each group. *p<0.01 versus Control. °p<0.01 versus CIA.

FIG. 11. This figure demonstrates the effect of combination therapy (DEX in μM+3 μM of M40401) on the LPS-stimulated TNF-α in LPS treated RAW cells.

FIG. 12. This figure demonstrates the effects of the oxidation product obtained from the reaction of dexamethasone with superoxide, tested in vitro for its ability to inhibit TNF-α production. The figure shows that the oxidation product has no activity on TNF-α.

FIG. 13. This figure demonstrates the effects of dexamethasone and FeTMPS ((5,10,15,20-tetrakis (2,4,6-trimethyl-3,5-disulfonatophenyl)-porphyrinate iron (III)) in carrageenan-induced paw edema. The results show that a low dose of FeTMPS (1 mg/kg) (note: mg/kg is also expressed as mpk) when combined with low dose of Dexamethasone (0.1 mg/kg) enhances the effects of Dexamethasone such that the combination dosage is equivalent to giving a higher dose of 3 mg/kg of Dexamethasone.

### ABBREVIATIONS AND DEFINITIONS

To facilitate understanding of the invention, a number of terms and abbreviations as used herein are defined below.

As used herein, the term “corticosteroid” refers to any of the adrenal corticosteroid hormones isolated from the adrenal cortex or produced synthetically, and derivatives thereof that are used for treatment of inflammatory diseases, such as arthritis, asthma, psoriasis, inflammatory bowel disease, lupus, and others. Corticosteroids include those that are naturally occurring, synthetic, or semi-synthetic in origin, and are characterized by the presence of a steroid nucleus of four fused rings, e.g., as found in cholesterol, dihydroxycholesterol, stigmasterol, and lanosterol structures. Corticosteroid drugs include cortisone, cortisol, hydrocortisone (11β, 17-dihydroxy, 21-(phosphonooxy)-pregn-4-ene, 3,20-dione disodium), dihydroxycortisone, dexamethasone (21-(acetyloxy)-9-fluoro-11β, 17-dihydroxy-16α-methylpregna-1,4-diene-3,20-dione), and highly derivatized steroid drugs such as beconase (beclomethasone dipropionate, which is 9-chloro-11β, 17,21, trihydroxy-16β-methylpregna-1,4 diene-3,20-dione 17,21-dipropionate). Other examples of corticosteroids include flunisolide, prednisone, prednisolone, methylprednisolone, triamcinolone, deflazacort and betamethasone.

As used herein, the terms “reactive oxygen species” or “ROS” refers to a toxic or reactive superoxide anion (O₂⁻). The superoxide anion, as well as the nitric oxide (NO⁻) and the hydroxyl radical (OH⁻) are different types of free-radicals.

As used herein, the terms “non-peptidic catalysts for the dismutation of superoxide” or “non-proteinaceous catalysts for the dismutation of superoxide” mean a low-molecular weight catalyst for the conversion of superoxide anions into hydrogen peroxide and molecular oxygen. These catalysts commonly consist of an organic ligand and a chelated transition metal ion, preferably copper, manganese(II), manganese(III), iron(II) or iron(III). The term may include catalysts containing short-chain polypeptides (under 15 amino acids) or macrocyclic structures derived from amino acids, as the organic ligand. The term explicitly excludes a superoxide dismutase enzyme (SOD) obtained from any species.

The term “catalyst for the dismutation of superoxide” means any catalyst for the conversion of superoxide anions into hydrogen peroxide and molecular oxygen. The term explicitly includes a superoxide dismutase enzyme (SOD) obtained from any species.

The term “substituted” means that the described moiety has one or more substituents comprising at least 1 carbon or heteroatom, and further comprising 0 to 22 carbon atoms, more preferably from 1 to 15 carbon atoms, and comprising 0 to 22 heteroatoms, more preferably from 0 to 15 heteroatoms. As used herein, “heteroatom” refers to those atoms that are neither carbon nor hydrogen bound to carbon and are selected from the group consisting of: O, S, N, P, Si, B, F, Cl, Br, or I. These atoms may be arranged in a number of configurations, creating substituent groups which are unsaturated, saturated, or aromatic. Examples of such substituents include branched or unbranched alkyl, alkenyl, or alkynyl, cyclic, heterocyclic, aryl, heteroaryl, allyl, polycycloalkyl, polycycloaryl, polycycloheteroaryl, imines, aminoalkyl, hydroxyalkyl, hydroxyl, phenol, amine oxides, thioalkyl, carboalkoxyalkyl, carboxylic acids and their derivatives, keto, ether, aldehyde, amine, amide, nitrile, halo, thiol, sulfoxide, sulfone, sulfonic acid, sulfide, disulfide, phosphonic acid, phosphinic acid, acrylic acid, sulphonamides, amino acids, peptides, proteins, carbohydrates, nucleic acids, fatty acids, lipids, nitro, hydroxylamines, hydroxamic acids, thiocarbonyls, thiocarbonyls, borates, boranes, boraza, silyl, silaza, siloxy, and combinations thereof.

The term “alkyl”, alone or in combination, means a straight-chain or branched-chain alkyl radical containing from 1 to about 22 carbon atoms, preferably from about 1 to about 18 carbon atoms, and most preferably from about 1 to about 12 carbon atoms. Examples of such radicals include, but are not limited to, methyl, ethyl, n-propyl, isopropyl, n-butyl, isobutyl, sec-butyl, tert-butyl, pentyl, iso-amyl, hexyl, octyl, nonyl, decyl, dodecyl, tetradecyl, hexadecyl, octadecyl and eicosyl.

The term “alkenyl”, alone or in combination, means an alkyl radical having one or more double bonds. Examples of such alkenyl radicals include, but are not limited to, ethenyl, propenyl, 1-butenyl, cis-2-butenyl, trans-2-butenyl, iso-butylenyl, cis-2-pentenyl, trans-2-pentenyl, 3-methyl-1-butenyl, 2,3-dimethyl-2-butenyl, 1-pentenyl, 1-hexenyl, 1-octenyl, decenyl, dodecenyl, tetradecenyl, hexadecenyl, cis- and trans-9-octadecenyl, 1,3-pentadienyl, 2,4-pentadienyl, 2,3-pentadienyl, 1,3-hexadienyl, 2,4-hexadienyl, 5,8,11,14-eicosatetraenyl, and 9,12,15-octadecatrienyl.

The term “alkynyl”, alone or in combination, means an alkyl radical having one or more triple bonds. Examples of such alkynyl groups include, but are not limited to, ethynyl, propynyl (propargyl), 1-butynyl, 1-octynyl, 9-octadecynyl, 1,3-pentadiynyl, 2,4-pentadiynyl, 1,3-hexadiynyl, and 2,4-hexadiynyl.

The term “cycloalkyl”, alone or in combination means a cycloalkyl radical containing from 3 to about 10, preferably from 3 to about 8, and most preferably from 3 to about 6, carbon atoms. Examples of such cycloalkyl radicals include, but are not limited to, cyclopropyl, cyclobutyl, cyclopentyl, cyclohexyl, cycloheptyl, cyclooctyl, and perhydronaphthyl.

The term “cycloalkylalkyl” means an alkyl radical as defined above which is substituted by a cycloalkyl radical as defined above. Examples of cycloalkylalkyl radicals include, but are not limited to, cyclohexylmethyl, cyclopentylmethyl, (4-isopropylcyclohexyl)methyl, (4-t-butyl-cyclohexyl)methyl, 3-cyclohexylpropyl, 2-cyclohexylmethylpentyl,3-cyclopentylmethylhexyl, 1-(4-neopentylcyclohexyl) methylhexyl, and 1-(4-isopropylcyclohexyl)methylheptyl.

The term “cycloalkylcycloalkyl” means a cycloalkyl radical as defined above which is substituted by another cycloalkyl radical as defined above. Examples of cycloalkylcycloalkyl radicals include, but are not limited to, cyclohexylcyclopentyl and cyclohexylcyclohexyl.

The term “cycloalkenyl”, alone or in combination, means a cycloalkyl radical having one or more double bonds. Examples of cycloalkenyl radicals include, but are not limited to, cyclopentenyl, cyclohexenyl, cyclooctenyl, cyclopentadienyl, cyclohexadienyl and cyclooctadienyl.

The term “cycloalkenylalkyl” means an alkyl radical as defined above which is substituted by a cycloalkenyl radical as defined above. Examples of cycloalkenylalkyl radicals include, but are not limited to, 2-cyclohexen-1-ylmethyl, 1-cyclopenten-1-ylmethyl, 2-(1-cyclohexen-1-yl)ethyl, 3-(1-cyclopenten-1-yl)propyl, 1-(1-cyclohexen-1-ylmethyl)pentyl, 1-(1-cyclopenten-1-yl)hexyl, 6-(1-cyclohexen-1-yl) hexyl, 1-(1-cyclopenten-1-yl)nonyl and 1-(1-cyclohexen-1-yl)nonyl.

The terms “alkylcycloalkyl” and “alkenylcycloalkyl” mean a cycloalkyl radical as defined above which is substituted by an alkyl or alkenyl radical as defined above. Examples of alkylcycloalkyl and alkenylcycloalkyl radicals include, but are not limited to, 2-ethylcyclobutyl, 1-methylcyclopentyl, 1-hexylcyclopentyl, 1-methylcyclohexyl, 1-(9-octadecenyl)cyclopentyl and 1-(9-octadecenyl)cyclohexyl.

The terms “alkylcycloalkenyl” and “alkenylcycloalkenyl” means a cycloalkenyl radical as defined above which is substituted by an alkyl or alkenyl radical as defined above. Examples of alkylcycloalkenyl and alkenylcycloalkenyl radicals include, but are not limited to, 1-methyl-2-cyclopentyl, 1-hexyl-2-cyclopentenyl, 1-ethyl-2-cyclohexenyl, 1-butyl-2-cyclohexenyl, 1-(9-octadecenyl)-2-cyclohexenyl and 1-(2-pentenyl)-2-cyclohexenyl.

The term “aryl”, alone or in combination, means a phenyl or naphthyl radical which optionally carries one or more substituents selected from alkyl, cycloalkyl, cycloalkenyl, aryl, heterocycle, alkoxyaryl, alkaryl, alkoxy, halogen, hydroxy, amine, cyano, nitro, alkylthio, phenoxy, ether, trifluoromethyl and the like, such as phenyl, p-tolyl, 4-methoxyphenyl, 4-(tert-butoxy)phenyl, 4-fluorophenyl, 4-chlorophenyl, 4-hydroxyphenyl, 1-naphthyl, 2-naphthyl, and the like.

The term “aralkyl”, alone or in combination, means an alkyl or cycloalkyl radical as defined above in which one hydrogen atom is replaced by an aryl radical as defined above, such as benzyl, 2-phenylethyl, and the like.

The term “heterocyclic” means ring structures containing at least one heteroatom within the ring. As used herein, “heteroatom” refer to atoms that are neither carbon nor hydrogen bound to a carbon. Examples of heterocyclics include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups.

The term “saturated, partially saturated or unsaturated cyclic” means fused ring structures in which 2 carbons of the ring are also part of the fifteen-membered macrocyclic ligand. The ring structure can contain 3 to 20 carbon atoms, preferably 5 to 10 carbon atoms, and can also contain one or more other kinds of atoms in addition to carbon. The most common of the other kinds of atoms include nitrogen, oxygen and sulfur. The ring structure can also contain more than one ring.

The term “saturated, partially saturated or unsaturated ring structure” means a ring structure in which one carbon of the ring is also part of the fifteen-membered macrocyclic ligand. The ring structure can contain 3 to 20, preferably 5 to 10, carbon atoms and can also contain nitrogen, oxygen and/or sulfur atoms.

The term “nitrogen containing heterocycle” means ring structures in which 2 carbons and a nitrogen of the ring are also part of the fifteen-membered macrocyclic ligand. The ring structure can contain 2 to 20, preferably 4 to 10, carbon atoms, can be substituted or unsubstituted, partially or fully unsaturated or saturated, and can also contain nitrogen, oxygen and/or sulfur atoms in the portion of the ring which is not also part of the fifteen-membered macrocyclic ligand.

The term “organic acid anion” refers to carboxylic acid anions having from about 1 to about 18 carbon atoms.

The term “halide” means chloride, fluoride, iodide, or bromide.

As used herein, “R” groups means all of the R groups attached to the carbon atoms of the macrocycle, i.e., R, R′, R1, R′1, R2, R′2, R3, R′3, R4, R′4, R5, R′5, R6, R′6, R7, R′7, R8, R′8, R9, and R′9.

The “mammal patient” in the methods of the invention is a mammal suffering from inflammatory disease or disorder. It is envisioned that a mammal patient to which the catalyst for the dismutation of superoxide in combination with a corticosteroid will be administered, in the methods or compositions of the invention, will be a human. However, other mammal patients in veterinary (e.g., companion pets and large veterinary animals) and other conceivable contexts are also contemplated.

As used herein, the terms “treatment” or “treating” relate to any treatment of inflammatory disease or disorders and include: (1) preventing inflammatory disease from occurring in a subject; (2) inhibiting the progression or initiation of the inflammatory disease, i.e., arresting or limiting its development; or (3) ameliorating or relieving the symptoms of the inflammatory disease.

The term “inflammatory disease” or “inflammatory disorder” refers to any disease marked by inflammation, which may be caused by a multitude of inciting events, including radiant, mechanical, chemical, infections, and immunological stimuli. Some inflammatory diseases include, but are not limited to, arthritis, inflammatory bowel disease, asthma, psoriasis, organ transplant rejections, radiation-induced injury, cancer, lupus and other autoimmune disorders, bums, trauma, stroke, rheumatic disorders, renal diseases, allergic diseases, infectious diseases, ocular diseases, skin diseases, gastrointestinal diseases, hepatic diseases, cerebral edema, sarcoidosis, thrombocytopenia, spinal cord injury, autoimmune disorders, or any other disease of disorder that may be treated with corticosteroids.

The term “arthritis” refers to inflammation of the joints and refers to a group of more than 100 rheumatic diseases that cause joint swelling, tissue damage, stiffness, pain (both acute and chronic), and fever. Arthritis can also affect other parts of the body other than joints including but not limited to: synovium, joint space, collagen, bone, tendon, muscle and cartilage, as well as some internal organs. The two most common forms of arthritis are osteoarthritis (“OA”) and rheumatoid Arthritis (“RA”). RA is the most severe of these two forms in terms of pain; while OA is the most common form.

The term “precursor ligand” means the organic ligand of a SOD mimic without the chelated transition metal cation and charge neutralizing anions.

The term “therapeutically effective amounts” means those amounts that, when administered to a particular subject in view of the nature and severity of that subject's disease or condition, will have the desired therapeutic effect, e.g., an amount which will cure, or at least partially arrest or inhibit the disease or condition.

The term “joint” or “joints” refers to the place of union or junction between two or more bones of the skeleton.

The term “co-administration” shall mean the administration of at least two agents to a subject either simultaneously or sequentially so as to provide the beneficial effects of the combination of both agents.

All references cited herein are explicitly incorporated by reference.

### DESCRIPTION OF THE PREFERRED EMBODIMENT

The present invention is directed to methods and compositions for the prevention and treatment of inflammatory diseases comprising administering compositions containing a non-proteinaceous catalyst for dismutation of superoxide in sequence, as in at least two preparations, or in combination, as in at least one preparation, with a corticosteroid. The catalyst for the dismutation of superoxide and the corticosteroid can be administered to a subject sequentially in separate formulations, or simultaneously as a single preparation or as a separate formulation. The compositions of this invention may be administered to the subject subcutaneously, intravenously, or intramuscularly. In a preferred embodiment, the compositions of this invention are administered to a subject subcutaneously or intramuscularly.

Some corticosteroids useful for this invention include, but are not limited to, cortisol, cortisone, hydrocortisone fludrocortisone, prednisone, prednisolone, 6-methylprednisolone, triamcinolone, betamethasone, and dexamethasone. However, any of the adrenal corticosteroid hormones isolated from the adrenal cortex or produced synthetically, and derivatives thereof that are used for treatment of inflammation are useful for this invention.

As shown in the example below, one particular advantage of this invention is that the use of SOD mimics in combination with corticosteroids enhances the efficiency of the corticosteroids in the treatment of inflammatory diseases and thereby allowing the use of a lower dosage of corticosteroids and decreasing the risk of side effects associated with corticosteroids. Glucocorticoids and their receptors become deactivated when exposed to superoxide and other free radicals, thereby forcing an increase in the dosage of glucocorticoids to have the desired therapeutic effect. In fact, administration of antioxidants to LPS treated RAW cells prevents the inactivation of dexamethasone as shown in Example 2. The dosage of corticosteroid needed for treatment of inflammatory disease is decreased by at least about 1%, more preferably by at least 10%, even more preferably by at least 25%, and most preferably by at least 50% when used in combination with the catalysts for dismutation of superoxide of this invention. The synergism associated with the combined use of SOD mimics and corticosteroids provides strong advantages for the treatment of inflammatory diseases.

Preferably, the compound employed in the method of the present invention will comprise a non-proteinaceous catalyst for the dismutation of superoxide anions (“SOD mimic”) as opposed to a native form of the SOD enzyme. As utilized herein, the term “SOD mimic” means a low-molecular-weight catalyst for the conversion of superoxide anions into hydrogen peroxide and molecular oxygen. These catalysts consist of an organic ligand having a pentaazacyclopentadecane portion and a chelated transition metal ion, preferably manganese or iron. The term may include catalysts containing short-chain polypeptides (under 15 amino acids), or macrocyclic structures derived from amino acids, as the organic ligand. The term explicitly excludes a SOD enzyme obtained from any natural sources. SOD mimics are useful in the method of the present invention as compared to native SOD because of the limitations associated with native SOD therapies such as, solution instability, limited cellular accessibility due to their size, immunogenicity, bell-shaped dose response curves, short half-lives, costs of production, and proteolytic digestion. See, e.g., Salvemini et al., Science 286: 304-306 (1999). For example, the best known native SOD, CuZn, has a molecular weight of 33,000 kD. In Contrast, SOD mimics have an approximate molecular weight of 400 to 600 Daltons.

In a particularly preferred embodiment, the SOD mimics utilized in the present invention comprise an organic ligand chelated to a metal ion. Particularly preferred catalysts are pentaaza-macrocyclic ligand compounds, more specifically the copper, manganese(II), manganese (III), iron(II) and iron(III) chelates of pentaazacyclopentadecane compounds. The pentaaza macrocyclic ligand complexes of Mn(II) are particularly advantageous for use in the present invention because, in addition to having a low molecular weight, they are highly selective for the dismutation of superoxide anions and possess catalytic rates similar to or faster than native SOD counterparts. Examples of this class of SOD mimic, M40403 and M40401, are set forth in the examples below. These pentaazacyclopentadecane compounds can be represented by the following formnula:

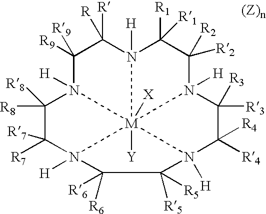

wherein M is a cation of a transition metal, preferably manganese or iron; wherein R, R′, R1, R′1, R2, R′2, R3, R′3, R4, R′4, R5, R′5, R6, R′6, R7, R′7, R8, R′8, R9, and R′9 independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R1 or R′1 and R2 or R′2, R3 or R′3 and R4 or R′4, R5 or R′5 and R6 or R′6, R7 or R′7 and R8 or R′8, and R9 or R′9 and R or R′ together with the carbon atoms to which they are attached independently form a substituted or unsubstituted, saturated, partially saturated or unsaturated cyclic or heterocyclic having 3 to 20 carbon atoms; R or R′ and R1 or R′1, R2 or R′2 and R3 or R′2, R4 or R′4 and R5 or R′5, R6 or R′6 and R7 or R′7, and R8 or R′8 and R9 or R′9 together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R1 and R′1, R2 and R′2, R3 and R′3, R4 and R′4, R5 and R′5, R6 and R′6, R7 and R′7, R8 and R′8, and R9 and R′9, together with the carbon atom to which they are attached independently form a saturated, partially saturated, or unsaturated cyclic or heterocyclic having 3 to 20 carbon atoms; and one of R, R′, R1, R′1, R2, R′2, R3, R′3, R4, R′4, R5, R′5, R6, R′6, R7, R′7, R8, R′8, R9 and R′9 together with a different one of R, R′, R1, R′1, R2, R′2, R3, R′3, R4, R′4, R5, R′5, R6, R′6, R7, R′7, R8, R′8, R9 and R′9 which is attached to a different carbon atom in the macrocyclic ligand may be bound to form a strap represented by the formula:

—(CH2)ₓ—M—(CH2)_{w}—L—(CH2)_{z}—I—(CH2)_{y}—

wherein w, x, y and z independently are integers from 0 to 10 and M, L and I are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof.

A preferred compound of this class of pentaaza-macrocyclic class is designated M40401 and is represented by the following formula:

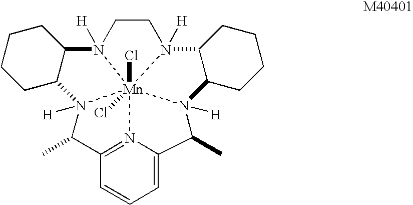

Another preferred compound of this class of pentaaza-macrocyclic class is designated M40403 and is represented by the following formula:

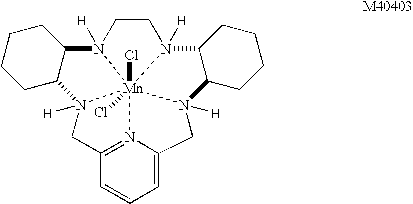

In another embodiment, the catalysts are substituted pentaaza-macrocyclic ligand compounds, which may be represented by the following formula:

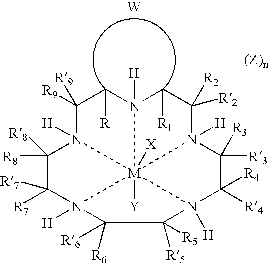

wherein a nitrogen of the macrocycle and the two adjacent carbon atoms to which it is attached independently form a substituted, unsaturated, nitrogen-containing heterocycle W having 2 to 20 carbon atoms, which may be an aromatic heterocycle, in which case the hydrogen attached to the nitrogen which is both part of the heterocycle and the macrocycle and the R groups attached to the carbon atoms which are both part of the heterocycle and the macrocycle are absent; and wherein R, R1, R2, R′2, R3, R′3, R4, R′4, R5, R′5, R6, R′6, R7, R′7, R8, R′8, R9, and R′9 independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; and, optionally, one or more of R2 or R′2 and R3 or R′3, R4 or R′4 and R5 or R′5, R6 or R′6 and R7 or R′7, or R8 or R′8 and R9 or R′9 together with the carbon atoms to which they are attached independently form a substituted or unsubstituted nitrogen containing heterocycle having 2 to 20 carbon atoms, which may be an aromatic heterocycle, in which case the hydrogen attached to the nitrogen which is both part of the heterocycle and the macrocycle and the R groups attached to the carbon atoms which are both part of the heterocycle and the macrocycle are absent; and, optionally, one or more of R2 and R′2, R3 and R′3, R4 and R′4, R5 and R′5, R6 and R′6, R7 and R′7, R8 and R′8, and R9 and R′9, together with the carbon atom to which they are attached independently form a saturated, partially saturated, or unsaturated cyclic or heterocyclic having 3 to 20 carbon atoms; and, optionally, one of R, R1, R2, R′2, R3, R′3, R4, R′4, R5, R′5, R6, R′6, R7, R′7, R8, R′8, R9, and R′9 together with a different one of R, R1, R2, R′2, R3, R′3, R4, R′4, R5, R′5, R6, R′6, R7, R′7, R8, R′8, R9, and R′9 which is attached to a different carbon atom in the macrocyclic ligand may be bound to form a strap represented by the formula:

—(CH2)ₓ—M—(CH2)_{w}—L—(CH2)_{z}—I—(CH2)_{y}—

wherein w, x, y and z independently are integers from 0 to 10 and M, L and I are independently selected from the group consisting of alkyl, alkenyl, alkynyl, aryl, cycloalkyl, heteroaryl, alkaryl, alkheteroaryl, aza, amide, ammonium, oxa, thia, sulfonyl, suffinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and combinations of any of the above; wherein M is a cation of a transition metal selected from the group consisting of manganese and iron; and wherein X, Y and Z represent suitable ligands or charge-neutralizing anions which are derived from any monodentate or polydentate coordinating ligand or ligand system or the corresponding anion thereof.

In a particularly preferred embodiment, the substituted pentaaza-macrocyclic ligand set forth above, W is a substituted pyridino moiety and U and V are trans-cyclohexanyl fused rings. In addition, the pentaaza-macrocyclic or substituted pentaaza-macrocyclic ligand compounds useful in the present invention can have any combinations of substituted or unsubstituted R groups, saturated, partially saturated or unsaturated cyclics, ring structures, nitrogen containing heterocycles, or straps as defined above.

X, Y and Z represent suitable ligands or charge-neutralizing anions which are derived from any monodentate or polydentate coordinating ligand or ligand system or the corresponding anion thereof (for example benzoic acid or benzoate anion, phenol or phenoxide anion, alcohol or alkoxide anion). X, Y and Z are independently selected from the group consisting of halide, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid(such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins. The preferred ligands from which X, Y and Z are selected include halide, organic acid, nitrate and bicarbonate anions.

The “R” groups attached to the carbon atoms of the macrocycle can be in the axial or equatorial position relative to the macrocycle. When the “R” group is other than hydrogen or when two adjacent “R” groups, i.e., on adjacent carbon atoms, together with the carbon atoms to which they are attached form a saturated, partially saturated or unsaturated cyclic or a nitrogen containing heterocycle, or when two R groups on the same carbon atom together with the carbon atom to which they are attached form a saturated, partially saturated or unsaturated ring structure, it is preferred that at least some of the “R” groups are in the equatorial position for reasons of improved activity and stability. This is particularly true when the complex contains more than one “R” group which is not hydrogen.

A wide variety of pentaaza-macrocyclic ligand compounds with superoxide dismutating activity may be synthesized. The transition metal center of the catalyst is thought to be the active site of catalysis, wherein the manganese or iron ion cycles between the (II) and (III) states.

The pentaaza-macrocyclic ligand compound catalysts described have been further described in U.S. Pat. Nos. 5,637,578, 6,214,817, and PCT application WO98/58636, all of which are hereby incorporated by reference. These pentaaza-macrocyclic ligand catalysts may be produced by the methods disclosed in U.S. Pat. No. 5,610,293.

Iron or manganese porphyrins are also suitable non-proteinaceous catalysts for use in the present invention, such as, for example, MnIII tetrakis(4-N-methylpyridyl)porphyrin, MnIII tetrakis-o-(4-N-methylisonicotinamidophenyl)porphyrin, MnIII tetrakis(4-N-N-N-trimethylanilinium)porphyrin, MnIII tetrakis(1-methyl4-pyridyl)porphyrin, MnIII tetrakis(4-benzoic acid)porphyrin, MnII octabromo-meso-tetrakis(N-methylpyridinium-4-yl)porphyrin, 5, 10, 15, 20-tetrakis (2,4,6-trimethyl-3,5-disulfonatophenyl)-porphyrinato iron (III) (FeTMPS), FeIII tetrakis(4-N-methylpyridyl)porphyrin, and FeIII tetrakis-o-(4-N-methylisonicotinamidophenyl)porphyrin and preferably, substituted iron porphyin 5,10,15,20-tetrakis (2,4,6-trimethyl-3,5-disulfonatophenyl)-porphyrinato iron (III) (FeTMPS) may also be used in the methods and compositions of the present invention which can be seen in U.S. Pat. No. 6,103,714, herein incorporated by reference in its entirety. The catalytic activities and methods of purifying or synthesizing these non-proteinaceous catalysts are well known in the organic chemistry arts.

Activity of the porphyrin compounds or complexes of the present invention for catalyzing the dismutation of superoxide can be demonstrated using the stopped-flow kinetic analysis technique as described in Riley, D. P. et al., Anal. Biochem., 196: 344-349 (1991) which is incorporated herein by reference. The stopped-flow kinetic analysis is suitable for screening compounds for SOD activity and activity of the porphyrin compounds or complexes of the present invention, as shown by stopped-flow analysis, correlate to treating the above disease states and disorders. However, the stopped-flow analysis is not an appropriate method for demonstrating the activity of all superoxide dismutase mimics. Other methods may be appropriate or preferred for some SOD mimics. See Weiss et al., Evaluation of Activity of Putative Superoxide Dismutase Mimics. Direct Analysis by Stopped-flow Kinetics, J.Biol.Chem. 268(31): 23049-54 (Nov. 5, 1993).

Contemplated equivalents of the general formulas set forth above for the compounds and derivatives as well as the intermediates are compounds otherwise corresponding thereto and having the same general properties such as tautomers of the compounds and such as wherein one or more of the various R groups are simple variations of the substituents as defined therein, e.g., wherein R is a higher alkyl group than that indicated, or where the tosyl groups are other nitrogen or oxygen protecting groups or wherein the 0-tosyl is a halide. Anions having a charge other than 1, e.g., carbonate, phosphate, and hydrogen phosphate, can be used instead of anions having a charge of 1, so long as they do not adversely affect the overall activity of the complex. However, using anions having a charge other than 1 will result in a slight modification of the general formula for the complex set forth above. In addition, where a substituent is designated as, or can be, a hydrogen, the exact chemical nature of a substituent which is other than hydrogen at that position, e.g., a hydrocarbyl radical or a halogen, hydroxy, amino and the like functional group, is not critical so long as it does not adversely affect the overall activity and/or synthesis procedure. Further, it is contemplated that manganese(III) complexes will be equivalent to the subject manganese(II) complexes.

For use in treatment or prophylaxis of subjects, the compounds of the invention can be formulated as pharmaceutical or veterinary compositions. Depending on the subject to be treated, the mode of administration, and the type of treatment desired (e.g., inhibition, prevention, prophylaxis, therapy), the compounds are formulated in ways consonant with these parameters. The compositions of the present invention comprise a therapeutically or prophylactically effective dosage of a catalyst for the dismutation of superoxide in combination with at least one corticosteroid. The catalyst for the dismutation of superoxide is preferably a SOD mimetic, as described in more detail above. The SODms of this invention, as well as the corticosteroids of this invention, are preferably used in combination with a pharmaceutically acceptable carrier, either in the same formulation or in separate formulations.

The compositions of the present invention may be incorporated in conventional pharmaceutical formulations (e.g., injectable solutions) for use in treating humans or animals in need thereof. Pharmaceutical compositions can be administered by subcutaneous, intravenous, or intramuscular injection, or as large volume parenteral solutions and the like. The term parenteral as used herein includes subcutaneous injections, intravenous, intramuscular, intrasternal injection, or infusion techniques.

For example, a parenteral therapeutic composition may comprise a sterile isotonic saline solution containing between 0.1 percent and 90 percent weight to volume of the catalysts for the dismutation of superoxide. A preferred solution contains from about 5 percent to about 25 weight percent catalysts for dismutation of superoxide in solution (% weight per volume). The parenteral therapeutic composition may contain, in addition to the isotonic saline solution and a catalyst for the dismutation of superoxide, at least one corticosteroid at between 1:100 to 100:1 weight ratio of the corticosteroid to the catalyst for the dismutation of superoxide. A preferred solution contains approximately 1:10 to 10:1 weight ratio of the corticosteroid to the catalyst for the dismutation of superoxide.

Alternatively, the corticosteroid may be administered sequentially to the catalyst for the dismutation of superoxide. The dosage of corticosteroid to be used may vary. A primary consideration for the dosage level of the corticosteroids of this invention is the monitoring of the known side effects in an individual.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The preparations may, if desired, be presented in a pack or dispenser device which may contain one or more unit dosage forms containing the catalyst for the dismutation of superoxide in conjunction with at least one corticosteroid. The pack may for example comprise metal or plastic foil, such as a blister pack. The pack or dispenser device may be accompanied by instructions for administration.

The invention also provides kits for carrying out the therapeutic regimens of the invention. Such kits comprise in one or more containers having therapeutically or prophylactically effective amounts of the catalyst and corticosteroid combination in pharmaceutically acceptable form the catalyst and corticosteroid combination in a vial of a kit of the invention may be in the form of a pharmaceutically acceptable solution, e.g., in combination with sterile saline, dextrose solution, or buffered solution, or other pharmaceutically acceptable sterile fluid. Alternatively, the complex may be lyophilized or desiccated; in this instance, the kit optionally further comprises in a container a pharmaceutically acceptable solution (e.g., saline, dextrose solution, etc.), preferably sterile, to reconstitute the complex to form a solution for injection purposes.

In another embodiment, a kit of the invention further comprises a needle or syringe, preferably packaged in sterile form, for injecting the combination, and/or a packaged alcohol pad. Instructions are optionally included for administration of the catalyst and corticosteroid combination by a clinician or by the patient.

The amount of active ingredient that may be combined with the carrier materials to produce a single dosage form will vary depending upon the host treated and the particular mode of administration. It will be appreciated that the unit content of active ingredients contained in an individual dose of each dosage form need not in itself constitute an effective amount, as the necessary effective amount could be reached by administration of a number of individual doses. The selection of dosage depends upon the dosage form utilized, the condition being treated, and the particular purpose to be achieved according to the determination of those skilled in the art.

The dosage regimen for treating a disease condition with the compounds and/or compositions of this invention is selected in accordance with a variety of factors, including the type, age, weight, sex, diet and medical condition of the patient, the route of administration, pharmacological considerations such as the activity, efficacy, pharmacokinetic and toxicology profiles of the particular compound employed, whether a drug delivery system is utilized and whether the compound is administered as part of a drug combination. Thus, the dosage regimen actually employed may vary widely and therefore may deviate from the preferred dosage regimen set forth above.

The pharmaceutical compositions of the present invention are preferably administered to a human. However, besides being useful for human treatment, these extracts are also useful for veterinary treatment of companion animals, exotic animals and farm animals, including mammals, rodents, avians, and the like. More preferred animals include horses, dogs, cats, sheep, and pigs.

The detailed description set forth above is provided to aid those skilled in the art in practicing the present invention. Even so, this detailed description should not be construed to unduly limit the present invention as modifications and variation in the embodiments discussed herein can be made by those of ordinary skill in the art without departing from the spirit or scope of the present inventive discovery.

All publications, patents, patent applications and other references cited in this application are herein incorporated by reference in their entirety as if each individual publication, patent, patent application or other reference were specifically and individually indicated to be incorporated by reference.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

### EXAMPLE 1

### Effects of Dexamethasone and M40403 in a Rodent Model of Collagen-Induced Arthritis

Objective. The objective of these studies were to determine whether low doses of M40403 potentiate the effects of low dose dexamethasone in the rat model of collagen induced arthritis.

Methods. Collagen-induced arthritis (CIA) was induced in Lewis rats by an intradermally injection of 100 μl of the emulsion (containing 100 μg of bovine type II collagen) (II) and incomplete Freund's adjuvant (IFA) at the base of the tail. On day 21, a second injection of CII in incomplete Freund's adjuvant was administered.

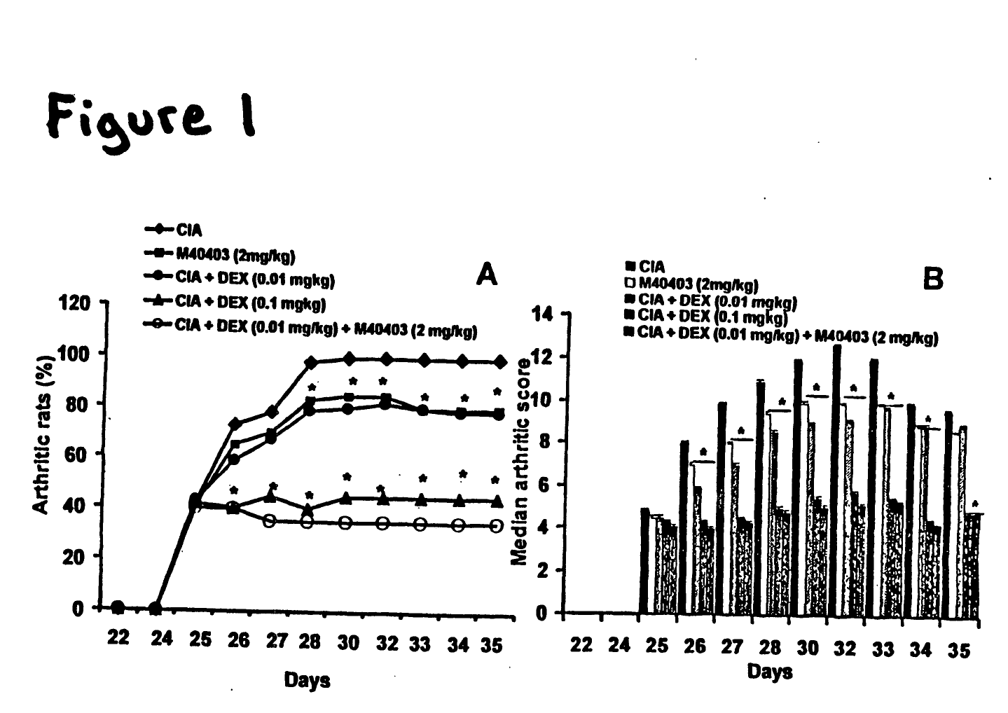

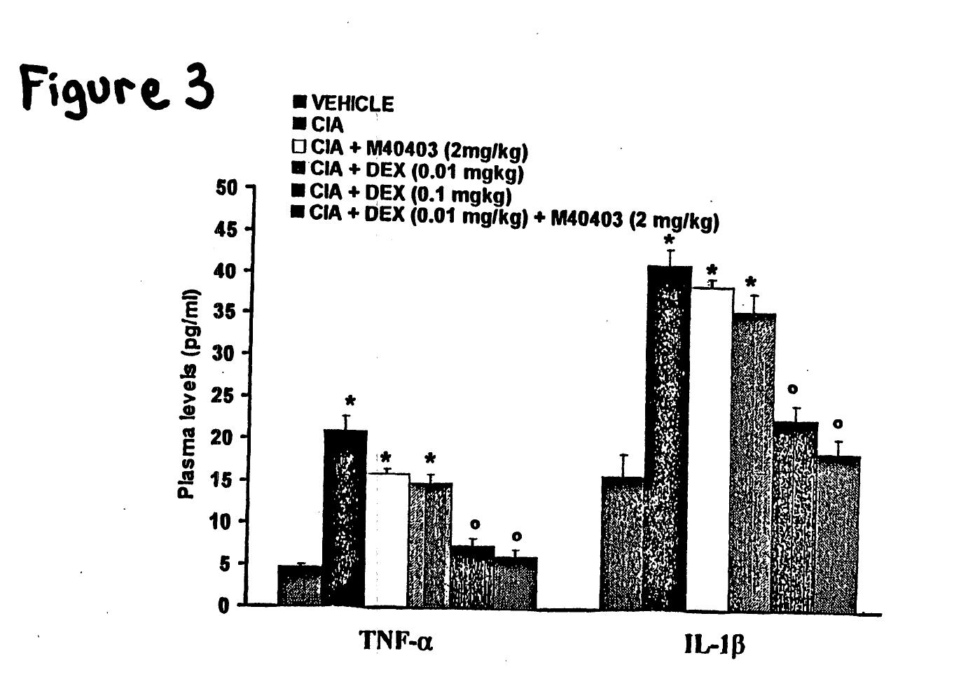

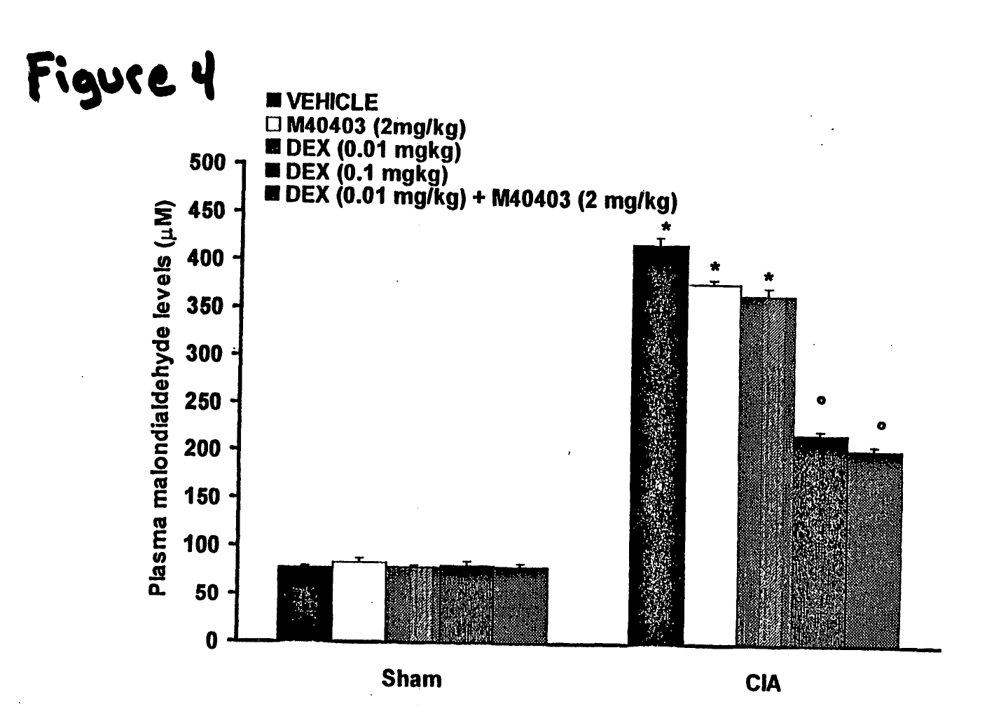

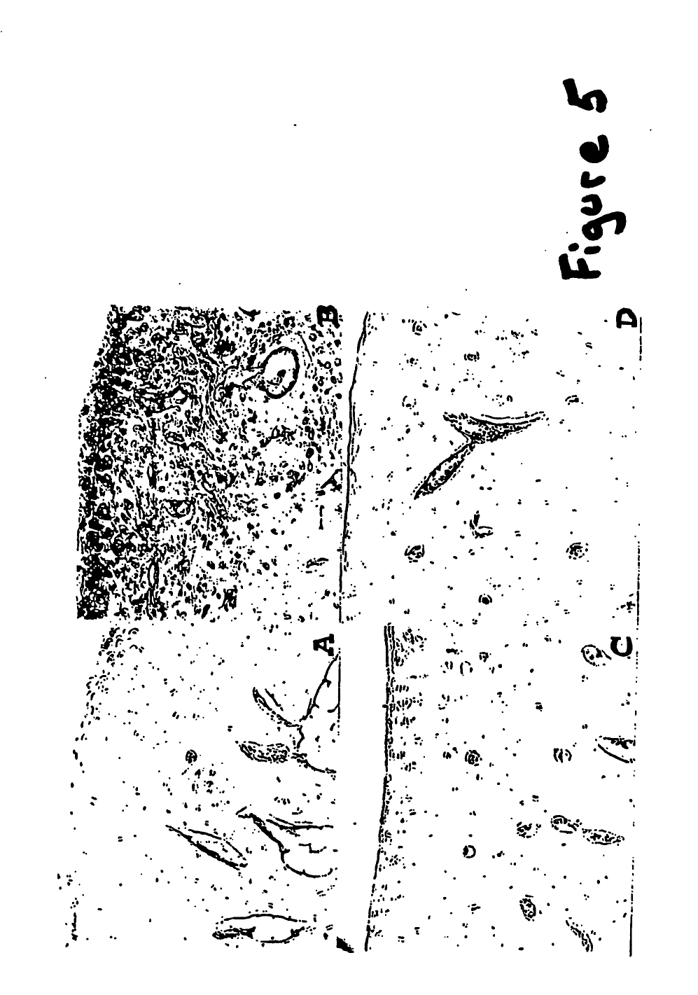

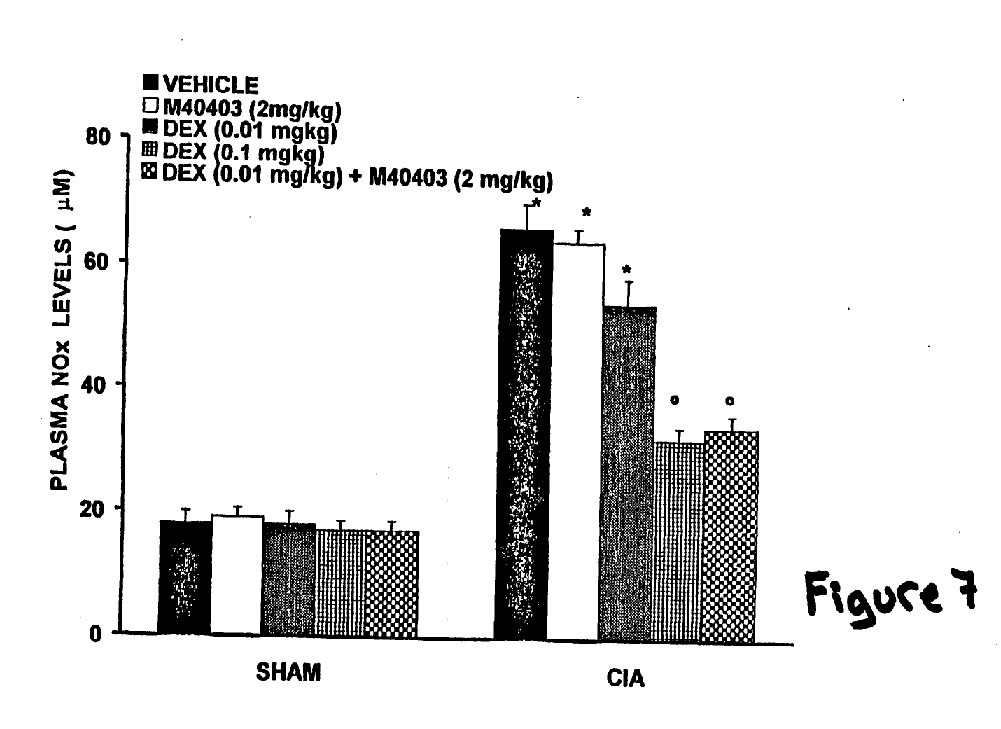

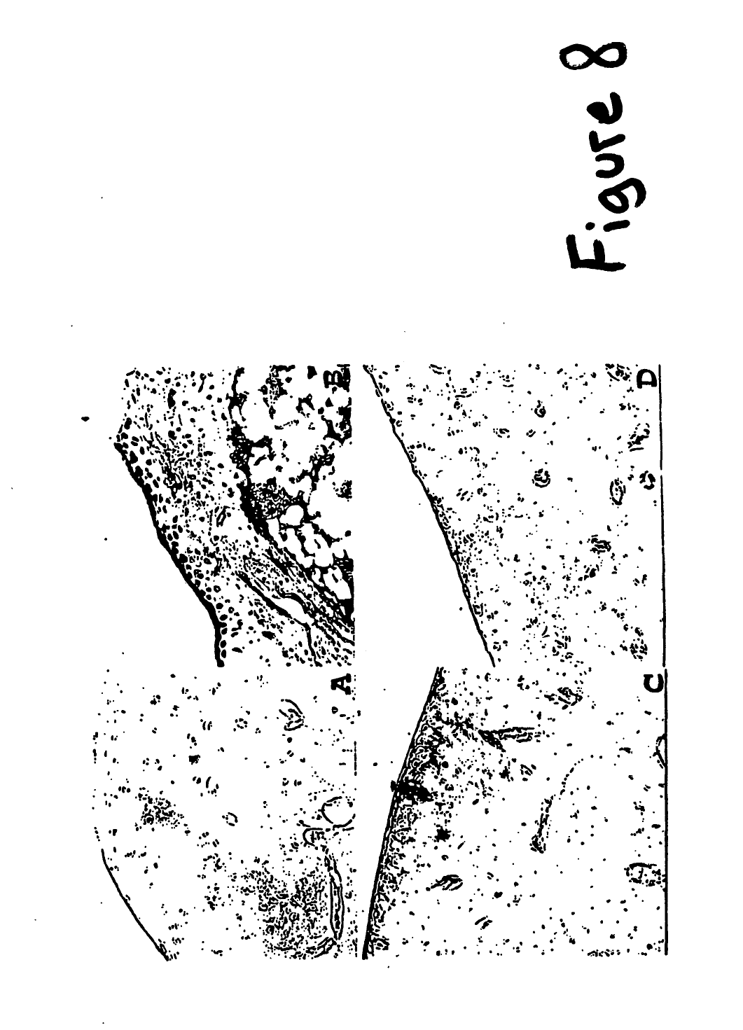

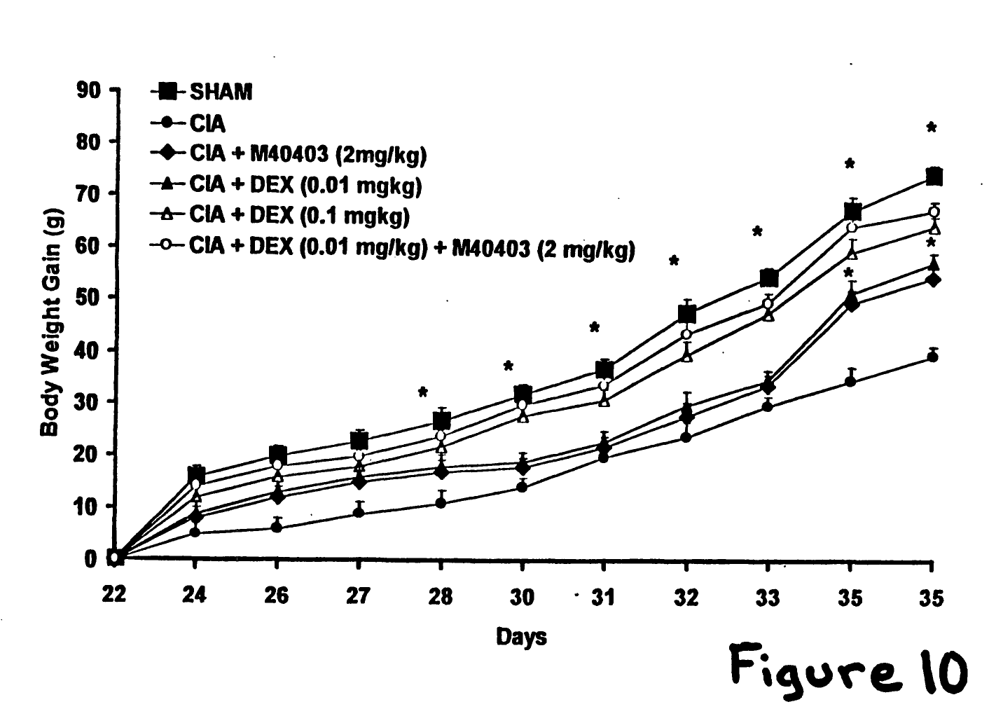

Results. Lewis rats developed an erosive hind paw arthritis when immunized with an emulsion of CII in IFA. Macroscopic clinical evidence of CIA first appeared as periarticular erythema and edema in the hind paws by day 24-26 after the first injection as shown in FIG. 1. The incidence of CIA was 100% by day 27 in the CII challenged rats; and CIA severity progressed over a 35-day period as shown in FIG. 1. A marked increase in the plasma levels of TNF-α and IL-1β as shown in FIG. 3, malonylaldehyde (MDA, a marker of lipid peroxidation) as shown in FIG. 4, and nitric oxide (NO) as shown in FIG. 7 was observed at day 35. Immunohistochemical analysis for nitrotyrosine (a marker for peroxynitrite formation) and PARS (a nuclear enzyme activated by DNA single strand damage) revealed a positive staining in inflamed joints from collagen-treated rats suggestive of the formulation of peroxynitrite and DNA damage as shown in FIG. 5. Immunohistochemical analysis for the inducible nitric oxide synthase and cyclooxygenase (iNOS and COX-2) revealed a positive staining in inflamed joints from collagen-treated rats as shown in FIG. 8. Treatment of rats with low does of M40403 (2 mg/kg daily, given by intraperitoneal injection) or a low dose of dexamethasone (0.01 mg/kg given daily orally) starting at the onset of arthritis (day 25), ameliorated the extent of the arthritic response (as defined by assessing the parameters described above) by some 10-20%. On the other hand, when these two low doses were combined the extent of the protective effects reached some 60-90%. The degree of protection observed with combination of these low doses was similar to the one attained with dexamethasone at 0.1 mg/kg. Finally, arthritic rats treated with combination of low doses of DEX (0.01 mg/kg) and M40403 (2 mg/kg) or with DEX at the high dosE (0.1 mg/kg) gained weight at the same rate and to the same extent as normal non-arthritic rats as shown in FIG. 10.

Conclusion. The study provides the first evidence that M40403, enhances the anti-inflammatory effects of dexamethasone in collagen-induced arthritis in the rat.

Methods

Animals

Male Lewis rats (weighing approximately 160-180 g and purchased from Charles River; Milan; Italy) were housed in a controlled environment and provided with standard rodent chow and water.

Experimental Protocol

Animals were randomly divided into six groups (n=10 for each group) as follows:

(1) Sham group: Rats received intraperitoneally (i.p.) a M40403 vehicle (26 mM sodium bicarbonate buffer, pH 8.1-8.3).

CIA groups: Rats were subjected to CIA as follows:

(2) CIA alone: In this group rats were subjected to CIA without receiving treatment with Compound A or DEX.

(3) CIA+M40403: In this group rats were subjected to CIA were treated with M40403 at 2 mg/kg i.p. every 24 hours, starting from day 25.

(4) CIA-DEX 0.01: In this group rats subjected to CIA were treated orally with Dexamethasone at 0.01 mg/kg starting from day 25.

(5) CIA+DEX 0.1: In this group rats subjected CIA were treated orally with Dexamethasone at 0.1 mg/kg starting from day 25.

(6) CIA+DEX+M40403: In this group rats subjected to CIA were treated with Dexamethasone (0.01 mg/kg, orally) and with M40403 (2 mg/kg, i.p.) starting from day 25.

Induction of Collagen-Induced Arthritis

Bovine type II collagen (CII) was dissolved in 0.01 M acetic acid at a concentration of 2 mg/ml by stirring overnight at 4° C. Dissolved CII was frozen at −70° C. until use. Incomplete Freund's adjuvant (IFA) was prepared by the addition of Mycobacterium tuberculosis H37Ra at a concentration of 2 mg/ml. Before injection, CII was emulsified with an equal volume of IFA. Collagen-induced arthritis was induced as previously described. On day 1, Lewis rats were injected intradermally at the base of the tail with 100 μl of the emulsion (containing 100 μg of CII). On day 21, a second injection of CII in IFA was administered.

Clinical Assessment of CIA

Rats were evaluated daily for arthritis by using a macroscopic scoring system: 0=no signs of arthritis; 1=swelling and/or redness of the paw or one digit; 2=two joints involved; 3=more than two joints involved; and 4=severe arthritis of the entire paw and digits. Arthritic index for each rat was calculated by adding the four scores of individual paws. Clinical severity was also determined by quantitating the change in the paw volume using plethysmometry (model 7140; Ugo Basile).

Immunohistochemical Localization of Nitrotyrosine, PARS, COX-2 and iNOS At day 35, the joints organs were then trimmed, placed in decalcifying solution for 24 hours and 8 μm sections were prepared from paraffin embedded tissues. After deparaffinization, endogenous peroxidase was quenched with 0.3% H₂O₂ in 60% methanol for 30 minutes. The sections were permeabilized with 0.1% Triton X-100 in PBS for 20 minutes. Non-specific adsorption was minimized by incubating the section in 2% normal goat serum in phosphate buffered saline for 20 minutes. Endogenous biotin or avidin binding sites were blocked by sequential incubation for 15 minutes with avidin and biotin. Sections were incubated overnight with 1) anti-rabbit polyclonal antibody directed at iNOS (1:1000 in PBS, v/v) (DBA, Milan, Italy) or 2) with anti-COX-2 goat policlonal antibody (1:500 in PBS, v/v) or 3) with anti-nitrotyrosine rabbit policlonal antibody (1:1000 in PBS, v/v) or 4) with anti-poly (ADP-Ribose) goat policlonal antibody rat (1:500 in PBS, v/v). Controls included buffer alone or non-specific purified rabbit IgG. Specific labelling was detected with a biotin-conjugated goat anti-rabbit IgG (for nitrotyrosine and iNOS) or with a biotin-conjugated goat anti-rabbit IgG (for PARS and COX-2) and avidin-biotin peroxidase complex. In order to confirm that the immunoreaction for the nitrotyrosine was specific some sections were also incubated with the primary antibody (anti-nitrotyrosine) in the presence of excess nitrotyrosine (10 mM) to verify the binding specificity. To verify the binding specificity for PARS, COX-2 and iNOS, some sections were also incubated with only the primary antibody (no secondary) or with only the secondary antibody (no primary). In these situations, no positive staining was found in the sections indicating that the immunoreaction was positive in all the experiments carried out.

Measurement of Nitrite/nitrate (NOₓ) Plasma levels of nitrite/nitrate (NOₓ) were measured as an indicator of NO synthesis. Briefly, the nitrate in the supernatant was first reduced to nitrite by incubation with nitrate reductase (670 mU/ml) and NADPH (160 μm) at room temperature for 3 hours. The nitrite concentration in the samples was then measured by the Griess reaction, by adding 100 μl of Griess reagent (0.1% naphthylethylendiamide dihydrochloride in H₂O and 1% sulphanilamide in 5% concentrated H₃PO₄; vol. 1:1) to 100 μl samples. The optical density at 55 nm (OD₅₅₀) was measured using ELISA microplate reader (SLT-Labinstruments Salzburg, Austria). Nitrate concentrations were calculated by comparison with OD₅₅₀ of standard solutions of DMEN.

Malondialdehyde (MDA) Measurement

Plasma malondialdehyde (MDA) levels were determined as an indicator of lipid peroxidation. An aliquot (100 μl) of the plasma collected at the specified time was added to a reaction mixture containing 200 μl of 8.1% SDS, 1500 μl of 20% acetic acid (pH 3.5), 1500 μl of 0.8% thiobarbituric acid and 700 μl distilled water. Samples were then boiled for 1 hour at 95° C. and centrifuged at 3,000×g for 10 minutes. The absorbance of the supernatant was measured by spectrophotometry at 650 nm.

Measurement of Cytokines TNF-α and IL-1 levels were evaluated in plasma at 35 days after the induction of arthritis. The assay was carried out by using a colorimetric, commercial kit (Calbaiochem-Novabiochem Corporation, USA). The ELISA has a lower detection limited of 5 pg/ml.

Materials

Unless otherwise stated, all compounds were obtained from Sigma-Aldrich Company Ltd. (Poole, Dorset, UK). Thiopentone sodium (Intraval Sodium®) was obtained from Rhone Merieux Ltd. (Harlow, Essex, UK). Biotin blocking kit, biotin-conjugated goat anti-rabbit IgG, Primary anti-nitrotyrosine, anti-poly (ADP-Ribose) synthetase antibodies primary anti-iNOS, anti-COX-2 and avidin-biotin peroxidase complex were obtained from DBA (Milan, Italy). All other chemicals were of the highest commercial grade available. All stock solutions were prepared in nonpyrogenic saline (0.9% NaCl; Baxter Healthcare Ltd., Thetford, Northfold, UK).

Data Analysis

Data analysis. All values in the figures and text are expressed as mean±standard error of the mean (s.e.m.) of n observations. For the in vivo studies, n represents the number of animals studied. In the experiments involving histology or immunohistochemistry, the figures shown are representative of at least three experiments performed on different experimental days. Data sets were examined by one- and two-way analysis of variance, and individual group means were then compared with Student's unpaired t test. For the arthritis studies, Mann-Whitney U test (two-tailed, independent) was used to compare medians of the arthritic indices (51). Values for the in vitro studies are presented as incidences (%), or medians. A p-value less than 0.05 was considered significant.

Results

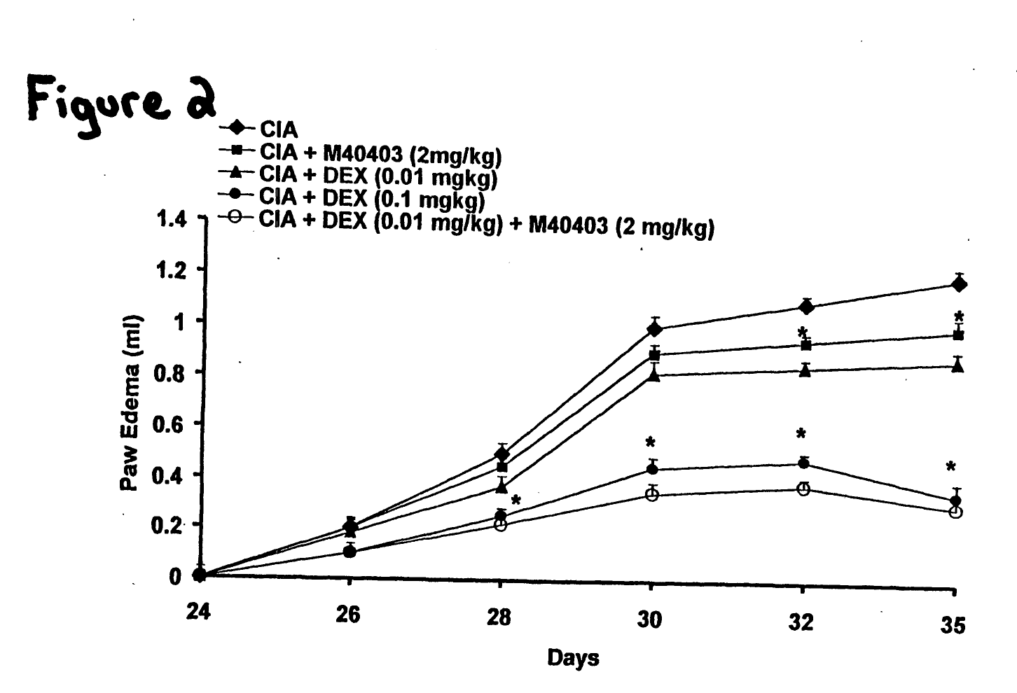

Effects of Combination Therapy in the Development of Collagen-Induced Arthritis CIA developed rapidly in rats immunized with CII and clinical signs (periarticular erythema and oedema) of the disease (FIG. 1A) first appeared in the hind paws between 24 and 26 days post-challenge. Furthermore, a 100% incidence of CIA was observed by day 28 in CII-immunized rats. Hind paw erythema and swelling increased in frequency and severity in a time-dependent mode with maximum arthritis indices of approximately 13 observed between 28 to 35 days post-immunization (FIG. 1B). When given at the low doses M40403 (2 mg/kg, i.p.) or DEX (0.01 mg/kg, p.o.) attenuated the development of CIA and arthritic score by some 10-20%. The maximum incidence of CIA in rats which received the high dose of DEX (0.1 mg/kg) was 45% (FIG. 1A, p<0.01). At this high dose, DEX (0.1 mg/kg) also exerted a significant suppression (p<0.01) of the arthritis index between days 26 and 35 post-CII immunization (FIG. 1B). In other words, the efficacy of a low dose of DEX at 0.01 mg/kg when given together with low dose of M40403 (2 mg/kg) was comparable to the efficacy of DEX at 0.1 mg/kg. Similar results were observed when assessing paw swelling (FIG. 2).

Effect of Combination Therapy of Cytokine Production and Lipid Peroxidation

At day 35, the levels of TNF-α and IL-1β were significantly elevated in the plasma from CIA-treated rats (FIG. 3). The degree of inhibition of TNF-α and IL-1β observed with a combination of low doses of DEX and M40403 (0.01 mg/kg and 2 mg/kg, respectively) was similar to that observed with DEX alone at the high dose (0.1 mg/kg) (FIG. 3). Similar results were observed when assessing plasma levels of MDA as an indicator of lipid peroxidation (FIG. 4).

Nitrotyrosine Formation and PARS Activation

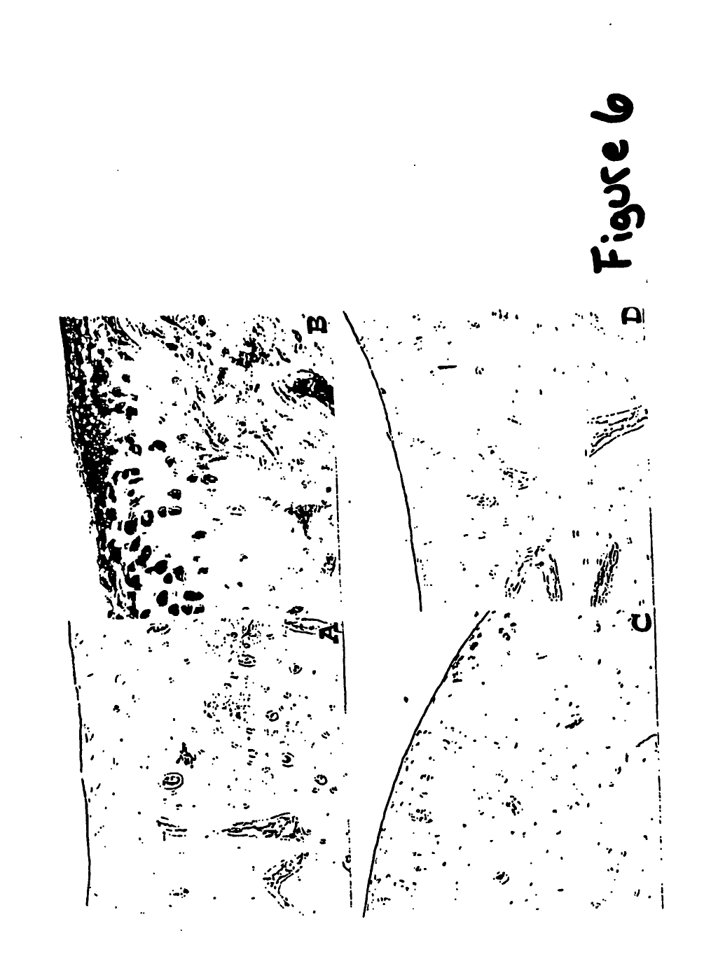

Immunohistochemical analysis and joint sections obtained from rats treated with collagen type II revealed a positive staining from nitrotyrosine and PARS, which was primarily localized in inflammatory cells (FIGS. 5B and 6B). No significant protective effect was observed in the group of animals treated with DEX (0.01 mg/kg) or with M40403 (2 mg/kg). In contrast, no positive nitrotyrosine or PARS staining was found in the joint of CIA-treated rats, which had been treated with the high dose of DEX alone (0.1 mg/kg; FIGS. 5C and 6C) or the combination therapy of low dose DEX and M40403, respectively (0.01 mg/kg+M40403 2 mg/kg; FIGS. 5D and 6D). There was no staining for either nitrotyrosine or PARS in joint obtained from sham-operated rats (FIGS. 5A and 6A).

Effect of Combination Therapy on NO Production

At day 35, the levels of NOₓ were significantly elevated in the plasma from CIA-treated rats (FIG. 7). DEX at the highest dose (0.1 mg/kg) or the combination of low doses of DEX and M40403 (0.01 mg/kg and 2 mg/kg respectively) (FIG. 7) inhibited NOₓ to the same extent.

iNOS and COX-2 Expression

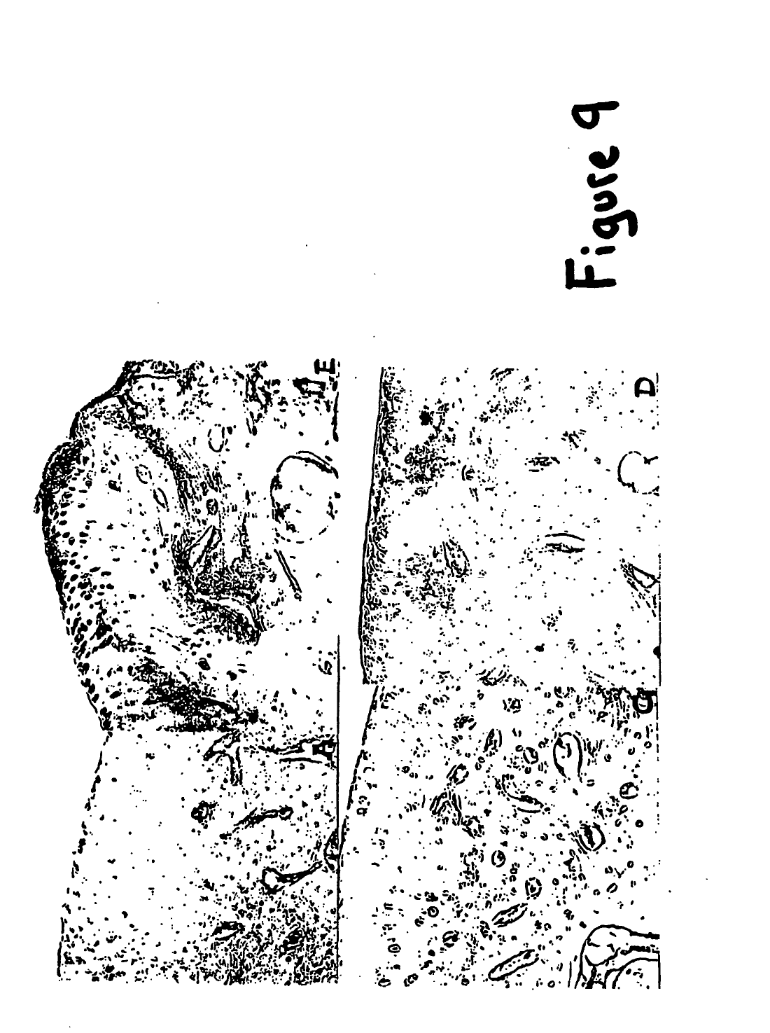

Immunohistochemical analysis of joint sections obtained from rats treated with collagen type II revealed a positive staining for iNOS, and COX-2 which was primarily localized in inflammatory cells (FIGS. 8B and 9B). In contrast, no positive iNOS or COX-2 staining was found in the joints of CIA-treated rats, which had been treated with high dose of DEX (0.1 mg/kg; FIGS. 8C and 9C) or the combination of low dose DEX and M40403 (0.01 mg/kg and 2 mg/kg respectively; FIGS. 8D and 9D). No staining for iNOS or COX-2 was observed in joint obtained from sham-operated rats (FIGS. 8A and 9A). DEX (0.01 mg/kg) or M40403 (2mg/kg) by themselves had no effect on iNOS or COX-2 staining.

Effects on Body Weight Gain

The rate and the absolute gain in body weight were comparable in sham Lewis rats and CII-immunized rats for the first week (FIG. 10). Beginning on day 25, the collagen-challenged rats gained significantly less weight than the normal rats, and this trend continued through day 35. Rats treated with the high dose DEX (0.1 mg/kg) or the combination of the low dose DEX and M40403 (0.01 mg/kg and 2 mg/kg M40403 respectfully) gained weight at a rate that was similar to the one observed with sham animals (FIG. 10). Rats treated with low doses DEX (0.01 mg/kg) or M40403 (2 mg/kg) gained weight in a manner that was similar to CIA rats (FIG. 10).

### EXAMPLE 2

### Biological Effect of the Use of Deacetylated Products of Dexamethasone and Cortisol Reacted with Reactive Oxygen Species

Objective. Two compounds were selected as model glucorticoids (dexamethasone and cortisol) for initial study. These were reacted with excess potassium superoxide in protic solvent to yield, upon purification, their respective C-17 deacetylated products.

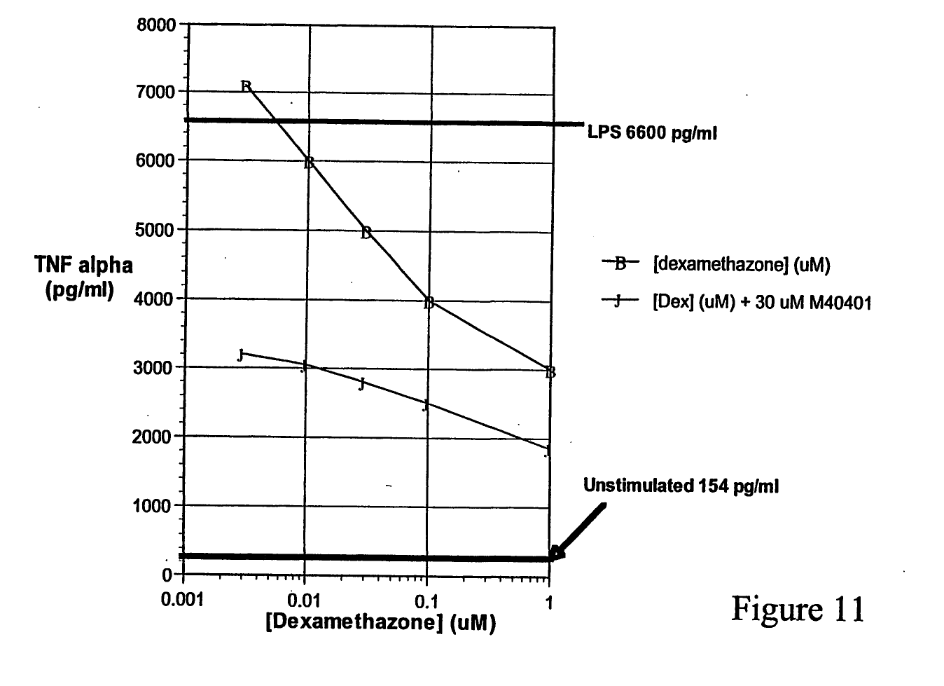

Methods. The biological effect of these products was then examined in vitro using the RAW macrophage cell line and whole blood assays. RAW cells are known to respond to LPS with an induction of iNOS and COX-2, as well as with a profound release of TNF-α release. Indeed, dexamethasone causes a dose-dependent inhibition of LPS-stimulated TNF-α as shown in FIG. 11.

FIG. 11 shows that administration of an antioxidant, the SOD mimic designated M40401, to LPS treated RAW cells enhances the effect of dexamethasone.

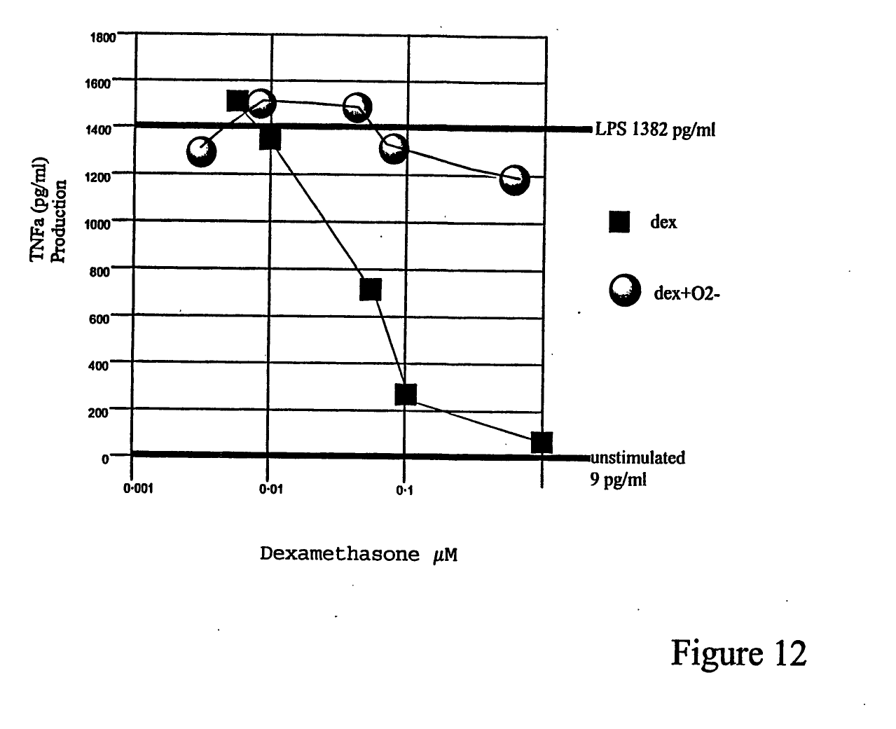

Remarkably, the presence of a superoxide dismutase mimic (M40401), at concentrations sufficiently below its own ability to inhibit the cytokine, causes a profound enhancement in the ability of dexamethasone to inhibit TNF-α. This suggests that LPS-activated macrophages release significant quantities of free radicals (e.g., superoxide and nitric oxide) which, in turn, affect the ability of dexamethasone to exert anti-inflammatory effects. Therefore, glucocorticoids that have been inactivated by free radicals would not be expected to depress nitric oxide, prostaglandin, or TNF-α production in in vitro or in vivo assays. In fact, the oxidation product obtained from the reaction of dexamethasone with superoxide, tested in vitro for its ability to inhibit TNF-α production, was found to have no activity as shown in FIG. 12.

Over the last few decades, there has been significant effort and accomplishment in the area of non-steroidal anti-inflammatory drugs (NSAIDs). NSAIDs (including nitric oxide synthase inhibitors and TNF-antibodies) exert their anti-inflammatory effects farther down the inflammation cascade than do glucocorticoids and they typically have fewer or less severe side effects. However, glucocorticoids were previously known to be the most potent anti-inflammatory agents. If a glucocorticoid could be produced having similar anti-inflammatory properties as dexamethasone while possessing diminished side effects, then inflammation could be mediated at the source of the cascade. The use of a combination therapy of SOD mimics and corticosteroids provides the efficient therapy of corticosteroids with diminished side effects because lower doses of corticosteroids in inflammation may be used.

### EXAMPLE 3

### Effects of Dexamethasone and FeTMPS in Carrageenan-Induced Paw Edema

Methods:

Dexamethasone was given by lavage one hour before carrageenan. FeTMPS (1 mg/kg) was given intravenously 15 minutes before carrageenan. Male sprague dawley rats weighing between 200 and 210 g were used. Paw edema was monitored for 6 hours. Results express delta change from basal. Each number is the mean+s.e.m. for n=4 rats per group.

Results

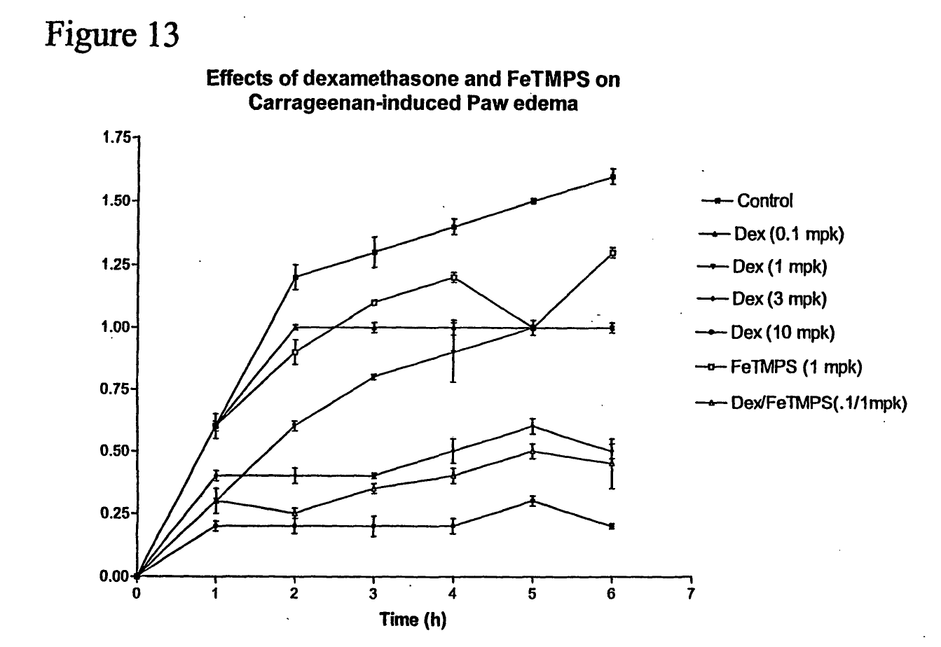

As can be seen from FIG. 13 and the Table 2 below, a low dose of FeTMPS (1 mg/kg) when combined with low dose dexamethasone (0.1 mg/kg) enhances the effects of dexamethasone. The combination of the compound and the low dose of dexamethasone (0.1 mg/kg) is as effective as a 3 mg/kg dose of dexamethasone.

TABLE 2
FeTMPS Effect with Dexamethasone

| Time (h) | | | | | | | Dex 0.1 mpk + |
|---|---|---|---|---|---|---|---|
| post | | Dex | Dex | Dex | Dex | FeTMPS | FeTMPS |
| carrageenan | Control | 0.1 mpk | 1 mpk | 3 mpk | 10 mpk | 1 mpk | 1 mpk |
| 0 | 0 | 0 | 0 | 0 | 0 | 0 | 0 |
| 1 | 0.6 ± 0.01 | 0.3 ± 0.05 | 0.3 ± 0.05 | 0.4 ± 0.02 | 0.2 ± 0.02 | 0.6 ± 0.02 | 0.3 ± 0.01 |
| 2 | 1.2 ± 0.05 | 1 ± 0.01 | 0.6 ± 0.02 | 0.4 ± 0.03 | 0.2 ± 0.03 | 0.9 ± 0.05 | 0.25 ± 0.02 |
| 3 | 1.3 ± 0.06 | 1 ± 0.02 | 0.8 ± 0.01 | 0.4 ± 0.01 | 0.2 ± 0.04 | 1.1 ± 0.01 | 0.35 ± 0.02 |
| 4 | 1.4 ± 0.03 | 1 ± 0.03 | 0.9 ± 0.012 | 0.5 ± 0.05 | 0.2 ± 0.03 | 1.2 ± 0.02 | 0.4 ± 0.03 |
| 5 | 1.5 ± 0.01 | 1 ± 0.01 | 1 ± 0.01 | 0.6 ± 0.03 | 0.3 ± 0.02 | 1 ± 0.03 | 0.5 ± 0.03 |
| 6 | 1.6 ± 0.03 | 1 ± 0.02 | 1 ± 0.02 | 0.5 ± 0.03 | 0.2 ± 0.01 | 1.3 ± 0.02 | 0.45 ± 0.1 |

In view of the above, it will be seen that the several objectives of the invention are achieved and other advantageous results attained.

1. A method for treating a subject afflicted with or susceptible to an inflammatory disease comprising co-administering a therapeutically effective amount to the subject of a catalyst for the dismutation of superoxide in conjunction with at least one corticosteroid.

2. A method according to claim 1, wherein the corticosteroid is selected from the group consisting of cortisol, cortisone, hydrocortisone, dihydrocortisone, fludrocortisone, prednisone, prednisolone, deflazacort, flunisolide, beconase, methylprednisolone, triamcinolone, betamethasone, and dexamethasone.

3. A method according to claim 1, wherein the corticosteroid is dexamethasone.

4. A method according to claim 1, wherein the corticosteroid is prednisone.

5. A method according to claim 1, wherein the catalyst is a non-proteinaceous catalyst, and the non-proteinaceous catalyst comprises an organic ligand chelated to a cation selected from the group of copper, manganese(II), manganese(III), iron(II) and iron(III).

6. A method according to claim 5, wherein the catalyst is a pentaaza-macrocyclic ligand complex or a substituted pentaaza-macrocyclic ligand complex.

7. A method according to claim 6, wherein the pentaaza-macrocyclic ligand complex is represented by the following formula: 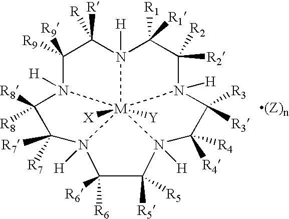
wherein
(a) R, R′, R₁, R′₁, R₂, R′₂, R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; and
(b) optionally, R₁ or R′₁ and R₂ or R′₂, R₃ or R′₃ and R₄ or R′₄, R₅ or R′₅ and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; and
(c) optionally, R or R′and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; and
(d) optionally, R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and
(e) optionally, one of R, R′, R₁, R′₁, R₂, R′₂, R₃, R′₃, R₄, R′₄, R₅, R′₅, R₆, R′₆, R₇, R′₇, R₈, R′₈, R₉, and R′₉ together with a different one of R, R′, R₁, R′₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula:
—(CH₂)ₓ—M—(CH₂)_{w}—L—(CH₂)_{z}—J—(CH₂)_{y}—
wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and
(f) combinations of any of (a) through (e) above; and wherein
X, Y and Z are independently selected from the group consisting of halide, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrite, aryl nitrite, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphino, aryl phosphino, alkyl phosphino oxide, aryl phosphino oxide, alkyl aryl phosphino oxide, alkyl phosphino sulfide, aryl phosphino sulfide, alkyl aryl phosphino sulfide, alkyl phosphonic acid, aryl phosphonic acid, alkyl phosphinic acid, aryl phosphinic acid, alkyl phosphinous acid, aryl phosphinous acid, phosphate, thiophosphate, phosphate, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins;
M is a cation of a transition metal, preferably manganese or iron, and
n is an integer from 0 to 3.

8. A method according to claim 6, wherein the substituted pentaaza-macrocyclic ligand complex is represented by the following formula: 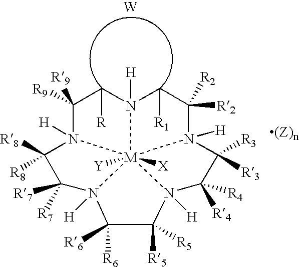
wherein
(a) a nitrogen of the macrocycle and the two adjacent carbon atoms to which it is attached independently form a substituted, unsaturated, nitrogen-containing heterocycle W having 2 to 20 carbon atoms, which may be an aromatic heterocycle, in which case the hydrogen attached to the nitrogen which is both part of the heterocycle and the macrocycle and the R groups attached to the carbon atoms which are both part of the heterocycle and the macrocycle are absent; and
(b) R, R₁, R₂, R′₂, R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; and
(c) optionally, one or more of R₂ or R′₂ and R₃ or R′₃, R₄ or R′₄ and R₅ or R′₅, R₆ or R′₆ and R₇ or R′₇, or R₈ or R′₈ and R₉ or R′₉ together with the carbon atoms to which they are attached independently form a substituted or unsubstituted nitrogen containing heterocycle having 2 to 20 carbon atoms, which may be an aromatic heterocycle, in which case the hydrogen attached to the nitrogen which is both part of the heterocycle and the macrocycle and the R groups attached to the carbon atoms which are both part of the heterocycle and the macrocycle are absent; and
(d) optionally, one or more of R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and
(e) optionally, one of R, R₁, R₂, R′₂, R₃, R′₃, R₄, R′₄, R₅, R′₅,R₆, R′₆, R₇, R′₇, R₈, R′₈, R₉, and R′₉ together with a different one of R, R₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula
—(CH₂)ₓ—M—(CH₂)_{w}—L—(CH₂)_{z}—J—(CH₂)_{y}—
wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and
(f) combinations of any of (a) through (e) above; and wherein
M is a cation of a transition metal selected from the group consisting of manganese and iron;
X, Y and Z represent suitable ligands or charge-neutralizing anions which are derived from any monodentate or polydentate coordinating ligand or ligand system or the corresponding anion thereof; and n is an integer from 0 to 3.

9. A method according to claim 8, wherein R₃ or R′₃ and R₄ or R′₄ together with the carbon atoms to which they are attached form a trans-cyclohexanyl fused ring and R₇ or R′₇ and R₈ or R′₈ together with the carbon atoms to which they are attached form a trans-cyclohexanyl fused ring.

10. A method according to claim 8, wherein W of the substituted pentaaza-macrocyclic ligand complex is a substituted pyridino moiety.

11. A method according to claim 8, wherein R₃ or R′₃ and R₄ or R′₄ together with the carbon atoms to which they are attached form a trans-cyclohexanyl fused ring; and R₇ or R′₇ and R₈ or R′₈ together with the carbon atoms to which they are attached form a trans-cyclohexanyl fused ring; and W is a substituted pyridino moiety.

12. A method according to claim 5, wherein the non-proteinaceous catalyst is a porphyrin ligand complex or a substituted porphyrin ligand complex.

13. A method according to claim 12, wherein the porphyrin ligand complex is selected from the group consisting of manganese(II) porphyrin complexes, manganese(III) porphyrin complexes, iron(II) porphyrin complexes, and iron(III) porphyrin complexes.

14. A method according to claim 13, wherein the porphyrin ligand complex is a 5,10,15,20-tetrakis (2,4,6-trimethyl-3,5-disulfonatophenyl)-porphyrinato iron(III) (FeTMPS) complex.

15. A method according to claim 1, wherein the subject is a mammal.

16. A method according to claim 15, wherein the mammal is a human.

17. A method according to claim 6, wherein the substituted pentaaza-macrocyclic ligand complex is represented by the following formula:

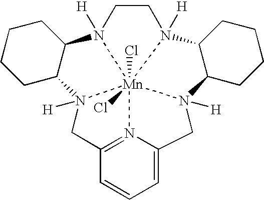

18. A method according to claim 6, wherein the substituted pentaaza-macrocyclic ligand complex is represented by the following formula:

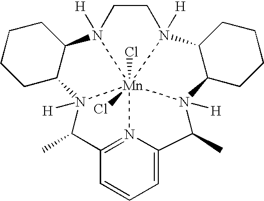

19. A method according to claim 1, wherein said co-administered corticosteroid is given in a dosage that is at least 50% less than the same corticosteroid administered alone to achieve said therapeutic effect.

20. A method according to claim 19, wherein said co-administered corticosteroid is given in a dosage that is at least 25% less than the same corticosteroid administered alone to achieve said therapeutic effect.

21. A method according to claim 20, wherein said co-administered corticosteroid is given in a dosage that is at least 10% less than the same corticosteroid administered alone to achieve said therapeutic effect.

22. A method according to claim 21, wherein said co-administered corticosteroid is given in a dosage that is at least 1% less than the same corticosteroid administered alone to achieve said therapeutic effect.

23. A method for treating a subject afflicted with or susceptible to arthritis comprising co-administering to the subject a therapeutically effective amount of a composition comprising a non-proteinaceous catalyst for the dismutation of superoxide anions and at least one corticosteroid.

24. A method according to claim 23, wherein the arthritis is rheumatoid arthritis.

25. A method according to claim 23, wherein the corticosteroid is selected from the group consisting of cortisol, cortisone, hydrocortisone, dihydrocortisone, fludrocortisone, prednisone, prednisolone, deflazacort, flunisolide, beconase, methylprednisolone, triamcinolone, betamethasone, and dexamethasone.

26. A method according to claim 23, wherein the corticosteroid is dexamethasone.

27. A method according to claim 23, wherein the corticosteroid is prednisone.

28. A method according to claim 23, wherein the non-proteinaceous catalyst comprises an organic ligand chelated to a metal ion selected from the group consisting of manganese(II), manganese(III), iron(II) and iron(III).

29. A method according to claim 28, wherein the non-proteinaceous catalyst is a pentaaza-macrocyclic ligand complex or a substituted pentaaza-macrocyclic ligand complex.

30. A method according to claim 29, wherein the pentaaza-macrocyclic ligand complex is represented by the following formula: 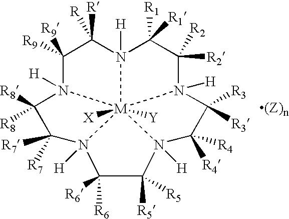
wherein
R, R′, R₁, R′₁, R₂, R′₂, R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; and
(b) optionally, R₁ or R′₁ and R₂ or R′₂, R₃ or R′₃ and R₄ or R′₄, R₅ or R′₅ and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; and
(c) optionally, R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; and
(d) optionally, R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and
(e) optionally, one of R, R′, R₁, R′₁, R₂, R′₂, R₃, R′₃, R₄, R′₄, R₅, R′₅, R₆, R′₆, R₇, R′₇, R₈, R′₈, R₉, and R′₉ together with a different one of R, R′, R₁, R′₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula:
—(CH₂)ₓ—M—(CH₂)_{w}—L—(CH₂)_{z}—J—(CH₂)_{y}—
wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and
(f) combinations of any of (a) through (e) above; and wherein
X, Y and Z are independently selected from the group consisting of halide, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrite, aryl nitrite, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfate bisulfite, thiosulfate, thiosulfite, hydrosulfite, alkyl phosphinc phosphino, aryl phosphino, alkyl phosphino oxide, aryl phosphino oxide, alkyl aryl phosphino oxide, alkyl phosphino sulfide, aryl phosphino sulfide, alkyl aryl phosphino sulfide, alkyl phosphonic acid, aryl phosphonic acid, alkyl phosphinic acid, aryl phosphinic acid, alkyl phosphinous acid, aryl phosphinous acid, phosphate, thiophosphate, phosphate, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins;
M is a cation of a transition metal, preferably manganese or iron; and
N is an integer from 0 to 3.

31. A method according to claim 29, wherein the substituted pentaaza-macrocyclic ligand complex is represented by the following formula: 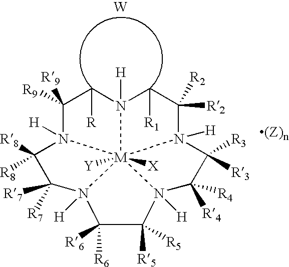
wherein
(a) a nitrogen of the macrocycle and the two adjacent carbon atoms to which it is attached independently form a substituted, unsaturated, nitrogen-containing heterocycle W having 2 to 20 carbon atoms, which may be an aromatic heterocycle, in which case the hydrogen attached to the nitrogen which is both part of the heterocycle and the macrocycle and the R groups attached to the carbon atoms which are both part of the heterocycle and the macrocycle are absent; and
(b) R, R₁, R₂, R′₂, R₃, R′₃, R₄, R′₄, R₅, R₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; and
(c) optionally, one or more of R₂ or R′₂ and R₃ or R′₃, R₄ or R′₄ and R₅ or R′₅, R₆ or R′₆ and R₇ or R′₇, or R₈ or R′₈ and R₉ or R′₉ together with the carbon atoms to which they are attached independently form a substituted or unsubstituted nitrogen containing heterocycle having 2 to 20 carbon atoms, which may be an aromatic heterocycle, in which case the hydrogen attached to the nitrogen which is both part of the heterocycle and the macrocycle and the R groups attached to the carbon atoms which are both part of the heterocycle and the macrocycle are absent; and
(d) optionally, one or more of R₂ and R′₂, R₃ and R′₃, R₄ and R′₄, R₅ and R′₅, R₆ and R′₆, R₇ and R′7, R₈ and R′8, and R₉ and R′₉, together with the carbon atom to which they are attached independently form a saturated, partially saturated, or unsaturated cyclic or heterocyclic having 3 to 20 carbon atoms; and
(e) optionally, one of R, R₁, R₂, R′₂, R₃, R′₃, R₄, R′₄, R₅, R′₅, R₆, R′_{6, R}₇, R′₇, R₈, R′₈, R₉, and R′₉ together with a different one of R, R₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula
—(CH₂)ₓ—M—(CH₂)_{w}—L—(CH₂)_{z}—J—(CH₂)_{y}—
wherein w, x, y and z independently are integers from 0 to 10 and M, L and j are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and
(f) combinations of any of (a) through (e) above; and wherein
M is a cation of a transition metal selected from the group consisting of manganese and iron;
X, Y and Z represent suitable ligands or charge-neutralizing anions which are derived from any monodentate or polydentate coordinating ligand or ligand system or the corresponding anion thereof; and
n is an integer from 0 to 3.

32. A method according to claim 31, wherein R₃ or R′₃ and R₄ or R′₄ together with the carbon atoms to which they are attached form a trans-cyclohexanyl fused ring and R₇ or R′₇ and R₈ or R′₈ together with the carbon atoms to which they are attached form a trans-cyclohexanyl fused ring.

33. A method according to claim 31, wherein W of the substituted pentaaza-macrocyclic ligand complex is a substituted pyridino moiety.

34. A method according to claim 31, wherein R₃ or R′₃ and R₄ or R′₄ together with the carbon atoms to which they are attached form a trans-cyclohexanyl fused ring; and R₇ or R′₇ and R₈ or R′₈ together with the carbon atoms to which they are attached form a trans-cyclohexanyl fused ring; and W is a substituted pyridino moiety.

35. A method according to claim 28, wherein the catalyst is a porphyrin ligand complex or a substituted porphyrin ligand complex.

36. A method according to claim 35, wherein the porphyrin ligand complex is selected from the group consisting of manganese (II) porphyrin complexes, manganese(III) porphyrin complexes, iron (II) porphyrin complexes, and iron(III) porphyrin complexes.

37. A method according to claim 36, wherein the porphyrin ligand complex is a 5,10,15,20-tetrakis (2,4,6-trimethyl-3,5-disulfonatophenyl)-porphyrinato iron (III) (FeTMPS).

38. A method according to claim 23, wherein the subject is a mammal.

39. A method according to claim 38, wherein the mammal is a human.

40. A method according to claim 29, wherein the substituted pentaaza-macrocyclic ligand complex is represented by the following formula:

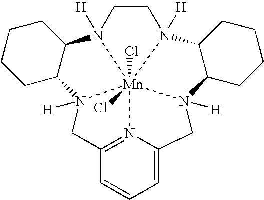

41. A method according to claim 29, wherein the substituted pentaaza-macrocyclic ligand complex is represented by the following formula:

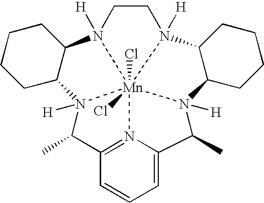

42. A method according to claim 23, wherein said co-administered corticosteroid is given in a dosage that is at least 50% less than the same corticosteroid administered alone to achieve said therapeutic effect.

43. A method according to claim 42, wherein said co-administered corticosteroid is given in a dosage that is at least 25% less than the same corticosteroid administered alone to achieve said therapeutic effect.

44. A method according to claim 43, wherein said co-administered corticosteroid is given in a dosage that is at least 10% less than the same corticosteroid administered alone to achieve said therapeutic effect.

45. A method according to claim 44, wherein said co-administered corticosteroid is given in a dosage that is at least 1% less than the same corticosteroid administered alone to achieve said therapeutic effect.

46. A pharmaceutical composition combination for the treatment of inflammatory disease comprising a non-proteinaceous catalyst for the dismutation of superoxide anions and a corticosteroid.

47. A combination according to claim 46, wherein the non-proteinaceous catalyst and corticosteroid together comprise a therapeutically effective amount of said non-proteinaceous catalyst and corticosteroid.

48. A combination according to claim 47, wherein the corticosteroid is selected from the group consisting of cortisol, cortisone, hydrocortisone, dihydrocortisone, fludrocortisone, prednisone, prednisolone, deflazacort, flunisolide, beconase, methylprednisolone, triamcinolone, betamethasone, and dexamethasone.

49. A combination according to claim 47, wherein the corticosteroid is dexamethasone.

50. A combination according to claim 47, wherein the corticosteroid is prednisone.

51. A combination according to claim 47, wherein the catalyst is a non-proteinaceous catalyst, and the non-proteinaceous catalyst comprises an organic ligand chelated to a cation selected from the group of copper, manganese (II), manganese (III), iron (II) and iron (III).

52. A combination according to claim 51, wherein the catalyst is a pentaaza-macrocyclic ligand complex or a substituted pentaaza-macrocyclic ligand complex.

53. A combination according to claim 52, wherein the pentaaza-macrocyclic ligand complex is represented by the following formula: 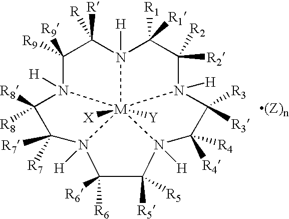
wherein M
(a) R, R′, R₁, R′₁, R₂, R′₂, R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; and
(b) optionally, R₁ or R′₁ and R₂ or R′₂, R₃ or R′₃ and R₄ or R′₄, R₅ or R′₅ and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; and
(c) optionally, R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; and
(d) optionally, R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and
(e) optionally, one of R, R′, R₁, R′₁, R₂, R′₂, R₃, R′₃, R₄, R′₄, R₅, R′₅, R₆, R′₆, R₇, R′₇, R₈, R′₈, R₉, and R′₉ together with a different one of R, R′, R₁, R′₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula:
—(CH₂)ₓ—M—(CH₂)_{w}—L—(CH₂)_{z}—J—(CH₂)_{y}—
wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, t+thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and
(f) combinations of any of (a) through (e) above; and wherein
X, Y and Z are independently selected from the group consisting of halide, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrite, aryl nitrite, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphino, aryl phosphino, alkyl phosphino oxide, aryl phosphino oxide, alkyl aryl phosphino oxide, alkyl phosphino sulfide, aryl phosphino sulfide, alkyl aryl phosphino sulfide, alkyl phosphonic acid, aryl phosphonic acid, alkyl phosphinic acid, aryl phosphinic acid, alkyl phosphinous acid, aryl phosphinous acid, phosphate, thiophosphate, phosphate, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins;
M is a cation of a transition metal, preferably manganese or iron; and
n is an integer from 0 to 3.

54. A combination according to claim 52, wherein the substituted pentaaza-macrocyclic ligand complex is represented by the following formula: 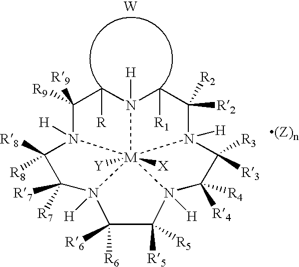
wherein
(a) a nitrogen of the macrocycle and the two adjacent carbon atoms to which it is attached independently form a substituted, unsaturated, nitrogen-containing heterocycle W having 2 to 20 carbon atoms, which may be an aromatic heterocycle, in which case the hydrogen attached to the nitrogen which is both part of the heterocycle and the macrocycle and the R groups attached to the carbon atoms which are both part of the heterocycle and the macrocycle are absent; and
(b) R, R₁, R₂, R′₂, R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; and
(c) optionally, one or more of R₂ or R′₂ and R₃ or R′₃, R₄ or R′₄ and R₅ or R′₅, R₆ or R′₆ and R₇ or R′₇, or R₈ or R′₈ and R₉ or R′₉ together with the carbon atoms to which they are attached independently form a substituted or unsubstituted nitrogen containing heterocycle having 2 to 20 carbon atoms, which may be an aromatic heterocycle, in which case the hydrogen attached to the nitrogen which is both part of the heterocycle and the macrocycle and the R groups attached to the carbon atoms which are both part of the heterocycle and the macrocycle are absent; and
(d) optionally, one or more of R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and
(e) optionally, one of R, R₁, R₂, R′₂, R₃, R′₃, R₄, R′₄, R₅, R′₅, R₆, R′₆, R₇, R′₇, R₈, R′₈, R₉, and R′₉ together with a different one of R, R₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula
—(CH₂)ₓ—M—(CH₂)_{w}—L—(CH₂)_{z}—J—(CH₂)_{y}—
wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and
(f) combinations of any of (a) through (e) above; and wherein
M is a cation of a transition metal selected from the group consisting of manganese and iron;
X, Y and Z represent suitable ligands or charge-neutralizing anions which are derived from any monodentate or polydentate coordinating ligand or ligand system or the corresponding anion thereof; and
n is an integer from 0 to 3.

55. A composition according to claim 54, wherein R₃ or R′₃ and R₄ or R′₄ together with the carbon atoms to which they are attached form a trans-cyclohexanyl fused ring and R₇ or R′₇ and R₈ or R′₈ together with the carbon atoms to which they are attached form a trans-cyclohexanyl fused ring.

56. A composition according to claim 54, wherein W of the substituted pentaaza-macrocyclic ligand complex is a substituted pyridino moiety.

57. A composition according to claim 54, wherein R₃ or R′₃ and R₄ or R′₄ together with the carbon atoms to which they are attached form a trans-cyclohexanyl fused ring; and R₇ or R′₇ and R₈ or R′₈ together with the carbon atoms to which they are attached form a trans-cyclohexanyl fused ring; and W is a substituted pyridino moiety.

58. A combinations according to claim 51, wherein the non-proteinaceous catalyst is a porphyrin ligand complex or a substituted porphyrin ligand complex.

59. A combinations according to claim 58, wherein the porphyrin ligand complex is selected from the group consisting of manganese(II) porphyrin complexes, manganese(II) porphyrin complexes, iron(II) porphyrin complexes, and iron(III) porphyrin complexes.

60. A combinations according to claim 59, wherein the porphyrin ligand complex is a 5,10,15,20-tetrakis (2,4,6-trimethyl-3,5-disulfonatophenyl)-porphyrinato iron(III) (FeTMPS) complex.

61. A combination according to claim 52, wherein the substituted pentaazamacrocyclic ligand complex is represented by the following formula:

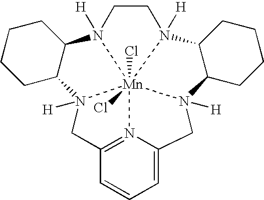

62. A combinations according to claim 52, wherein the substituted pentaazamacrocyclic ligand complex is represented by the following formula:

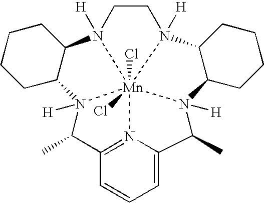

63. A kit comprising at least one non-proteinaceous catalyst and at least one corticosteroid.


SEQUENCE LISTING (1 sequence)

SEQ ID NO: 1 - protein, 154 aa, Homo sapiens [sequence omitted]